(19) United States
(12) Patent Application Publication
(10) Pub. No.: US 2009/0131346 A1
(43) Pub. Date: May 21, 2009

(54) ANTIBODY AND METHOD FOR IDENTIFICATION OF DENDRITIC CELLS

(75) Inventor: Claudio Sorio, Verona (IT)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: CONSORZIO PER GLI STUDI UNIVERSITARI IN VERONA, Verona (IT)

(21) Appl. No.: 11/572,098

(22) PCT Filed: Jul. 15, 2005
(86) PCT No.: PCT/IB2005/002027

§ 371 Date: Jan. 12, 2007

(30) Foreign Application Priority Data
Jul. 16, 2004 (IT) VI2004A000174

(51) Int. Cl. A61K 31/7088; C12Q 1/42; G01N 33/573; C12Q 1/68; C07K 16/18; G06N 5/02; C40B 40/08; C40B 30/04; C40B 40/10

(52) U.S. Cl. 514/44; 435/21; 435/7.4; 435/6; 530/387.9; 530/387.3; 506/17; 506/9; 506/18; 706/46

(57) ABSTRACT

Method for identifying myeloid or plasmacytoid dendritic cells provided by a mammal, stimulated or unstimulated, comprising the steps of: a) preparing a cell sample; b) contracting the cell sample myeloid or plasmacytoid dendritic cells to form a complex; c) detecting the complex; characterized in that the phosphatase is the Receptor type Tyrosine Phosphatase Gamma Protein (PTPRG), acting as a specific marker of said dendritic cells, and in that the compound is a polypeptide capable of selectively bind to the PTPRG or to a fragment thereof or to a oligonucleotide complementary to a PTPRG mRNA logonucleotide in such a manner as to allow the selective recognizing of the dendritic cells in the cell sample.

### FIELD OF THE INVENTION

The present invention relates to a method for identifying dendritic cells (hereinafter also indicated as DCs), either in vitro or in vivo, in steady state or following immunomodulatory treatments.

The invention finds application in the diagnostic and medical field for the selection of substances or pharmaceutical preparation for the modulation of the immunitary activity of the dendritic cells and for the identification of a normal or pathological state.

An another embodiment of the invention relates to an antibody and a chip on solid phase for identification, manipulation and stimulation of the dendritic cells, as well for measurement of their activity.

### BACKGROUND ART

Mechanisms relating to the acquired immunity involve several identification phases and reactions which involve several types of cells.

In this field operate dendritic cells (DC), which are named in function of their capability of emitting and retracting several and thin cytoplasmatic process similar to dendritic neurons. Initially confused by Paul Langerhans in 1968 with neurons, they are finally recognized by Ralph Steinman and Zanvil Cohn (Steinman, Lustig et al. 1974).

Dendritic cells (DC) represent a specific subset of APC that has a central role in the initiation and regulation of immune responses in both lymphoid and non-lymphoid tissues.

They are capable of take up antigens in tissues and migrate to lymphoid organs where they initiate an immune response interacting mainly with T cells.

This function is provided thanks to their ubiquitous distribution in the whole organism.

Depending on their localization, they are classified in: dendritic cells of blood (Caux, Dezutter-Dambuyant et al. 1992); tissues DCs, including Langerhans skin cells, (Bruynzeel-Koomen, van Wichen et al. 1986), respiratory system DCs (Fokkens, Broekhuis-Fluitsma et al. 1991), mucous membrane of digestive system DCs (Barrett, Cruchley et al. 1996); indeterminate cells of the skin and of the lamine propria of the mucous (Fokkens, Vinke et al. 1998) and the submucous (Soler, Tazi et al. 1991); veiled cells of lymphatic afferents (Balfour, Drexhage et al. 1981), interdigitating cells of regional lymph nodes (Balfour, Drexhage et al. 1981) and lymphoid structures of the mucous (Havenith, Breedijk et al. 1993); interstitial cells of organs, such as kidney, intestine, lung, thyroid gland (Sminia, Wilders et al. 1983); thymic dendritic cells (Ardavin 1997).

The existence of separate lineages of DCs is justified by the necessity of specific and non redundant functions, as suggested by the identification of DC subsets (myeloid and lymphoid) in mice (Banchereau e Steinman 1998) that exert different influences on the type of immune response generated in vivo (Maldonado-Lopez, De Smedt et al. 1999; Pulendran, Smith et al. 1999). This appear to be the case in humans as well (Res, Martinez-Caceres et al. 1996; Rissoan, Soumelis et al. 1999).

The key role of DC cells is evident as T cells recognize antigen only if this is a MHC-II/peptide complex. Actually, activation of native T cells needs three independent signals and the bond of the peptide-MHC complex, recognized by the T-cell receptor (TCR), transfers first signal to the T cells.

Second signal is of the co-stimulating type and is mediated by recognizing CD80/86, expressed because of the signal transferred from CD28 lymphocyte receptor to the toll like receptor TLR. Third signal is the release of soluble messengers, like as IL-12, promoting the differentiation of T cells in Th1 or Th2 cells.

### Ontogeny

The early development steps of DC formation from hematopoietic progenitor cells are not uniform and involve various types of progenitor cells, different developmental pathways and different signals.

Beginning from the first demonstration of the medullar origin of dendritic cells (Katz, Tamaki et al. 1979), several experiments are carried out to identify their precursors in the bone marrow and in blood and precursors of Langerhans cells. Among these, two principal ways have been followed.

The first way, described by Caux (Caux, Dezutter-Dambuyant et al. 1992), provides a system able to generate dendritic cells, similar to Langerhans CD1a+ cells, from staminal CD34+ cells with granulocyte/macrophage colony stimulating factor (GM-CSF) and tumor necrosis factor alpha (TNF-α).

Dendritic cells generation from Langerhans cells was improved in following experiments adding both/either stem cell factor (SCF) and/or FLT-3 ligand that provides a greater production of CD1a+ cells, having typical dendritic structure and great expression of antigen of class II, CD4, CD40, CD54, CD58, CD80, CD83 e CD56, and the production of Birbeck granules in 10-20% of the cells (Novak, Haberstok et al. 1999). These cells had a great ability to stimulate the production of virgin T cells and to provide antigens soluble in CD4+ lymphocytes clones.

The second way, described by Sallusto and Lanzavecchia (Sallusto and Lanzavecchia, 1994), provides the culture of monocytes with GM-CSF and interleucine-4 (IL-4) that generate in vitro CD1a+ dendritic cells similar, as phenotype, to interstitial dendritic cells. CD14+ monocytes are different from CD1a+ dendritic cells as they are Birbeck granules—free and they express CD11b, CD68 e XIIIa coagulation factor.

Typically, after 7 days of culture with GM-CSF and IL-4, monocytes provide immature dendritic cells that need further stimuli, e.g. with CD40 ligand (CD40L), with bacterial lipopolysaccharide (LPS), with endotoxin or TNF-α, to reach the maturation stage and become well stimulating dendritic cells.

### Heterogeneity of Dendritic Cells

In humans, DCs comprise an heterogeneous cellular population; this difference can be detected by observing the precursors, their anatomic localization, the function exerted proliferation of B cells or differentiation of T cells in Th1 o Th2) and the type of immune response induced (tolerance or immunity).

About phenotypic characterization, dendritic cells lack of lineage-specific markers such as CD3, CD14, CD15, CD16, CD19, CD20 and CD56, and are defined as “lineage negative” (lin-) cells (Lotze and Thomson 2001).

Despite of their importance in the immune system, dendritic cells are considered “lacking” from the ontogenetic point of view, because of their uncertain classification in a hematopoietic line (Peters, Gieseler et al. 1996).

Actually, in the dendritic cell system, it is possible to point out a lot of phenotypic and functional heterogeneities, that mark the difference between the maturation and the activation stage.

Furthermore, it is known that several surface markers are present both in lymphocytes and in dendritic cells; it point out that there are common thymic precursors. Monocytes also are able to generate dendritic cells (DCs) in presence of GM-CSF and IL-4.

Despite of affinities, dendritic cells and monocytes can also develop independently in myeloid cellular growing manner (Steinman and Inaba 1999). Moreover, skin dendritic cells express several markers as receptors for Fc, ATPase and non specific esterases, such as macrophages.

Several studies have brought to the identification of a number of marks useful for the determination of surface phenotype, intracellular markers and gene expressed selectively or preferentially by human DCs among which CCR7, fascin, CD68, DC-LAMP. Since they can be detected in macrophages in various experimental conditions, none of these markers is absolutely specific for DCs.

At present it is still unclear the way in which myeloid and lymphoid progenies are related.

Table 1 provides a model of hematopoietic differentiation. In every column are indicated specific markers of cells indicated upper in the same column.

TABLE 1
model of hematopoietic differentiation

| | LYMPHOID | | | |
|---|---|---|---|---|
| | origin | MYELOID origin | | |
| | Plasmacytoid | interstitial | Langerhans | monolite |
| Cellular lines | DCs | DCs | cells | derivate DCs |
| Phenotypes blood's | CD14⁻ | CD14⁺ | CD14⁺ | CD14⁺ |
| precursors | CD123⁺ | CD123⁻ | CD123⁻ | CD123⁻ |
| | CD11c⁻ | CD11c⁺ | CD11c⁺ | CD11c⁺ |
| | CD1a⁻ | CD1a⁻ | CD1a⁺ | CD1a⁺ |
| | ILR3⁺ | ILR3⁻ | ILR3⁻ | ILR3⁻ |
| Cytokines inducing | IL-3 | GM-CSF + IL4 | GM-CSF + IL4 | GM-CSF + IL4 |
| differentiation immature | | TGF-β | TGF-β | |
| DCs | | | | |
| Maturative stimulus | CD40-L | LPS, CD40-L, | CD40-L | LPS, CD40-L |
| | | MCM | | |
| Phenotype mature DCs | CD11b⁻ | CD11b⁺ | CD11b⁺ | CD11b⁺ |
| | CD13⁻ | CD13⁺ | CD13⁺ | CD13⁺ |
| | CD33⁻ | CD33⁺ | CD33⁺ | CD33⁺ |
| | Birbeck⁻ | Birbeck⁻ | Birbeck⁺ | Birbeck⁻ |
| | granules | granules | granules | granules |
| | Langerin⁻ | Langerin⁻ | Langerin⁺ | Langerin⁻ |
| | XIIIa⁻ | XIIIa⁻ | XIIIa⁻ | XIIIa⁻ |
| | MHC II class | MHC II class | MHC II class | MHC II class |
| | DC-LAMP⁺ | DC-LAMP⁺ | DC-LAMP⁺ (if | DC-LAMP⁺ |
| | | | mature) | |
| | DC-SIGN⁺ | DC-SlGN⁺ | DC-SIGN⁻ | DC-SIGN⁺ (⁺⁺ if |
| | | | | mature) |
| | CCR7⁺ | CCR7⁺ | CCR7⁺ (if | CCR7⁺ |
| | | | mature) | |
| | ? | Decysin⁺ | Decysin+ | Decysin+ |
| | CD 80⁺ e CD | CD 80⁺ e CD | CD 80⁺ CD 86⁺ | CD 80⁺ CD 86⁺ |
| | 86⁺ | 86⁺ | (if mature) | |
| Localization | T zone | T zone | T zone | T zone |
| | Lymphoid | Lymphoid | Lymphoid | Lymphoid |
| | organs | organs | organs | organs |
| | Tymus gland | and germinative | Mucous | Blood |
| | Blood | centers | membrane and | |
| | | Mucous | skin | |
| | | membrane and | Blood | |
| | | skin | | |
| | | Blood | | |
| Functions: | | | | |
| IL-12 secretion | +/− | ++++ | ++++ | ++++ |
| IFNa/β production | ++++ | − | − | − |
| CD4+ lymphocyte | ++ | ++++ | ++++ | ++++ |
| stimulus | | | | |
| CD8+ lymphocyte | ++ | +++ | ++++ | ++++ |
| stimulus | | | | |
| B DCs interaction | ? | ++++ | + | ? |
| Th1 lymphocyte different. | ++/− | ? | ? | ++++ |
| Th2 lymphocyte different. | ++ | ? | ? | +/− |

As a result, the ontogeny and interrelationship of human DC subsets require further investigation, and the identification of markers capable of identify and differentiate various subsets and/or differentiation stages is crucial in order to unravel this issue.

A known method for isolating DCs involves the elimination of cell lines positive for a specific marker, such as CD3, CD19 and CD56, and the positive selection of 123-CDs and MHCs-II. Thanks to this method, it is possible isolate plasmacytoid DCs, which are characterized in that they are positive for CD4, CD11a, CD18, CD32, CD36, CD38, CD40, CD44, CD45RA, CD49d, CD54, CD58, CD62L, CD95, CD123, and MHC-II.

However, myeloid DCs are characterized in that they are positive for CD1a/b/c CD4, CD11a, CD11c, CD13, CD18, CD29, CD31, CD32, CD33, CD36, CD38, CD40, CD44, CD45RO, CD49e, CD80, CD83 (after maturation), CD86, CD95, Mannose receptor CMRF-44 and MHC-II, and in that they are negative or subpositive for CD10, CD123, CD45RA, E-caderine, CLA (cutaneous lymphocyte-associated antigen), CD49d, CD21, CD2, CD5, CD7, CD25, CD62L, CD127.

For clinical uses is very important the achievement of immature DC populations overexpressing receptors for antigens.

At present, this aim is not achievable because of the lack of markers selectively expressed by immature DCs.

Actually, prior art teaches that it needs more then one marker for the identification of DCs.

However, in the methods having the object of stimulating or inhibiting DC's activity, it would be better to use a single marker and, preferably, a specific marker. Moreover, it would be preferable a synthetic or natural ligand of this marker, including an antibody or a fragment of the same.

According to this invention, a DC specific marker is a marker expressed by a dendritic cell. Finally, the DC identification by a single specific marker has not been heretofore possible.

### SUMMARY OF THE INVENTION

A main object of the present invention is that of providing a method for the identification of myeloid and plasmacytoid dendritic cells.

Another object is that of providing polypeptides acting as synthetic or natural ligands of a specific DC marker.

A further object of the invention is that of providing oligonucleotides complementary to oligonucleotides of the mRNA of a specific DC marker.

Another object of the present invention is that of providing a solid phase chip either for the identification of myeloid and plasmacytoid dendritic cells, in resting or activated state, or to confirm the identity and the maturation stage of the mDCs.

Moreover, an object of the invention is that of providing a method both/either for isolate and/or remove myeloid or plasmacytoid dendritic cells form a cell population.

Last but not least, an object of the invention is that to providing a method for the identification of an agent capable of definite the differentiation of cells to the DC phenotype.

These objects, together with others which will appear more clearly below, are achieved by a method for identifying myeloid and plasmacytoid dendritic cells, in accordance with claim 1, comprising the following steps: preparing a cell sample; contacting the cell sample with at least a compound capable of selectively bind to a phosphatase of the myeloid or plasmacytoid dendritic cells to form a complex; detecting the complex.

According to the invention, the phosphatase is the Receptor type Tyrosine Phosphatase Gamma Protein PTPRG, acting as a specific marker of the dendritic cells, and the compound is a polypeptide capable of selectively bind to the PTPRG or to a fragment thereof or to a oligonucleotide complementary to a PTPRG mRNA oligonucleotide in such a manner as to allow the selective recognizing of the dendritic cells in the cell sample.

Thanks to this method, it will be possible to identify dendritic cells from a cell population in a quick, low-cost and effective manner.

### BRIEF DESCRIPTION OF THE FIGURES

FIG. 1 shows a theoretical model of the development of DCs from a stem cell line of haematopoietic cells (HSC: haematopoietic stem cell) providing DC's myeloid (CMP) and lymphoid (CLP) precursors. The development provides the myeloid differentiation in blood DCs, non-epithelial DCs, Langerhans DCs, interdigital DCs and the lymphoid differentiation in plasmacytoid DCs (Nature Review Immunology).

FIG. 2 shows the results of a Western blotting with an anti-P4 antibody against a cell line treated to secrete extracellular PTPRG (spot 3 and 4). Spot 1 and 2: the same cell line transferted with a vacuum plasmid (recognizing specificity test) does not reacts.

FIG. 3 shows the cytofluorimetric analysis of the K562 cell line transferted with a “full length” PTPRG plasmid, and relative test, treated with anti-P4 peptide antibody. The latter provide a specific reactivity in cells expressing PTPRG.

FIG. 4 shows the expression of PTPRG in human blood and hemopoietic tissues:

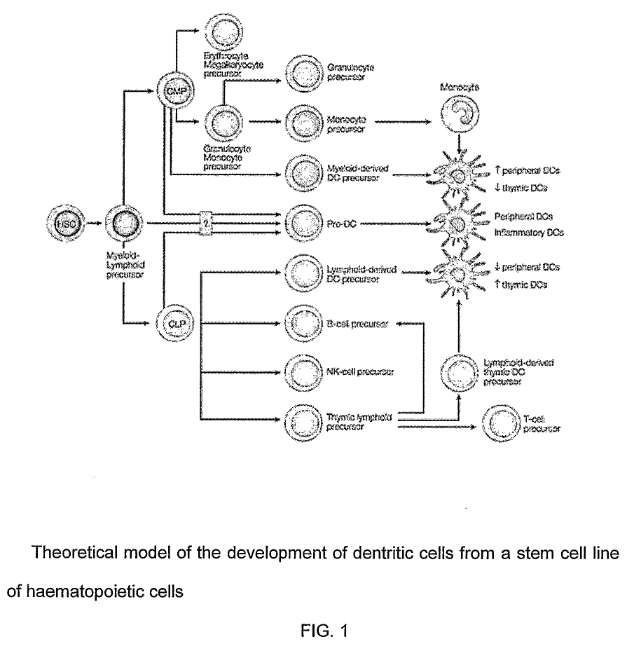

Panel A, upper: amplification of cDNA specific for PTPRG; lane 1: Plasmid containing PTPRG cDNA (positive control), lane 2: Lymphocytes, lane 3: purified PMNs, lane 4: Ficoll-purified MNC from bone marrow aspirate, lane 5: Thymus, lane 6: spleen, lane 7: U937. Lower panel lane 2 to 7: βactin expression in the corresponding cell and tissue samples demonstrate the presence of similar amounts of cDNA in all the lanes.

Panel B: In situ hybridization (ISH) shows the presence of PTPRG: specific RNA (antisense probe) in cells surrounding small vessels in spleen (40×). Control (sense probe) show no staining (insert).

Panel C: IHC using PTPRG-specific antibody C18 shows the presence of large, irregularly shaped cells rich in dendrites (brown) around arterioles and within white and red pulp in spleen (20×) such as in panel B.

Panels D, H: presence of reactivity for PTPRG in epithelial skin cells and dermic DCs detected by IHC (panel D).

Panels E, F: In situ hybridization (ISH) shows the presence of PTPRG reacting cells in thymus also (20×). A detail of panel E(F) shows a strong staining of irregularly shaped cells surrounded by thymocytes that do not express PTPg transcript (100×).

Panel G: IHC using PTPRG-specific antibody C18 showing the presence of dendritic cells within medullary and cortical areas of human thymus, note Hassal's bodies negative for PTPg staining and the presence of large, irregularly shaped cells corresponding to the elements shown in panel E and F.

FIG. 5 shows the localization of PTPRG reactivity in thymus and their phenotype characteristics. The upper panel shows an artistic representation of the structure and the thymic cell components to facilitate the interpretation of next images.

Panel A: PTPRG positive cells are present in the medulla (M) as well as in the cortical area (C). Positive cells (brown) displayed fusate/dendritic morphology but, in immunofluorescence (PTPRG green) did not react with markers related to the well known thymic DC population including plasmacytoid DC: CD123, red (Panel B) and mature DC: DCLAMP, red (Panel C). Significantly, as for secondary lymphoid organs, PTPRG stained a subset DCSIGN+ cells, located in the cortex (Panel D, arrow) confirming a preferential expression on monocyte-derived DC.

FIG. 6 shows double-staining analyses that demonstrate a different staining pattern between PTPRG positive cells, lymphocytes and cytokeratin positive epithelial cells.

FIG. 7 shows the PTPRG reactivity in lymph nodes and tonsils.

Panel A: reactivity within the secondary B-cell follicles was restricted to large non-lymphoid cells (CD20-CD3-) with abundant cytoplasm some of which containing phagocytized debris and thus corresponding to tingible body macrophages (Panel A: insert). Double immunofluorescence demonstrate coexpression of CD11c (Panel B). A population of PTPRG cells with clear dendritic morphology, was additionally identified in B-cell follicles (Panel C) and coexpress CD11c (red) and CD4 (green) (Panel C insert). Lack of CD21 and CD35 (Panel D) excluded their follicular dendritic cells identity. Double immunofluorescence analysis revealed a preferential clustering of germinal centre T-lymphocytes (red) around PTPg+cells (green) (Panel D, insert). Distribution of PTPRG+cells was not limited to B-cell compartment; since this protein was expressed on DCSIGN+ sinus macrophages (Panel E) and DCSIGN+ cells in the interfollicular area (Panel F) that lack CD1a and Langerin.

FIG. 8 shows the PCR analysis of the PTPRG expression in peripheral blood derived cells.

Panel A: Time course of PTPRG expression in monocytes after plating. Lane 1: freshly isolated monocytes. Lane 2: monocytes after 2 hours of culture. Lane 3: monocytes after 4 hours of culture. Lane 4: monocytes after 12 hours of culture. Lane 5: monocytes after 24 hours of culture. Lane 6: monocytes after 5 days of culture. Lane 7: monocytes after 5 days of culture in the presence of IL4 and GM-CSF (iMDDCs). Upper panel: PTPRG expression (arrow), lower panel: actin expression. One experiment representative of three individual donors

Panel B: Role of IL4 and GM-CSF on PTPRG expression. M: Molecular weight marker; Lane 1: freshly isolated monocytes. Lane 2: monocytes after 5 days of culture with no cytokines (macrophages). Lane 3: monocytes after 5 days of culture with GM-CSF. Lane 4: monocytes after 5 days of culture with IL-4. Lane 5: monocytes after 5 days of culture with GM-CSF and addition of IL-4 starting from day 3. Lane 6: monocytes after 5 days of culture in the presence of IL4 and GM-CSF (iMDDCs). Upper panel: PTPRG expression (arrow), lower panel: actin expression (one experiment representative of three individual donors).

FIG. 9 shows the PTPRG expression in monocytes, macrophages and DCs.

Panel A: PCR quantitative analysis of levels of mRNA for PTPRG in myeloid cells derived from peripheral blood and DCs treated with LPS in relation to the DC control (=1). DC=monocytes cultured with GM-CSF and IL-4 for 5 days. Macrophages: monocytes cultured in complete medium without cytokines for 6 days.

Panel B: IFNg affect DC differentiation. The figure and the table illustrate the fold-induction of CD14, CD1a, CD83, HLA-DR and PTPRG in relation to immature DCs (value=1). The numbers are derived from the measurement of mean fluorescence intensity. No expression of CD14 was detected (value=0).

FIG. 10 shows the negative reaction of macrophages in vivo.

Panel A: foreign-bodies macrophages negative to the stimulus with anti-PTPRG antibody. Look at the positive control of the dendritic/macrophages cells of the marginal sinus.

Panel B: asterisks indicate epitheloid cells from granulomatous lymphadenitis caused by a tuberculosis microbacteria infection.

FIG. 11 shows the PTPRG expression in comparison to a selection of DC-specific markers and receptor-like phosphatases in resting and activated MDMs and DCs. Lane 1: marker, Lane 2: Resting macrophages, Lane 3: Macrophages activated with LPS 100 ng/ml for 24 hours, Lane 4: Resting DCs, Lane 5: DCs activated with LPS 100 ng/ml for 24 hours. The genes analyzed are indicated on the left side of the picture.

FIG. 12 shows the expression of PTPRG in monocyte derived DCs analyzed by confocal microscopy. DC were left untreated or treated with LPS for 24 h and then stained with control antibody (Rabbit (RBT) IgG), anti PTPRG affinity purified polyclonal antibody P4 (PTPRG), followed by anti RBT IgG PE (red), FITC conjugated anti MHCII and anti CD83 antibodies (green). Inserts show the double staining of PTPRG expressing cells (red) with MHC II (second and third columns) and CD83 (last column, LPS treated cells) all in green. The presence of the yellow colour indicates co-localization of the selected proteins.

FIG. 13 shows the mRNA amplification specific for PTPRG and for a control gene (G6PDH): animals treated with LPS, even though for only 3 h, show an increased reaction to PTPRG compared to the control gene.

### DETAILED DESCRIPTION OF THE INVENTION

As will appear more clearly in the “MATERIAL and METHODS” section, the Receptor type Tyrosine Phosphatase Gamma Protein, hereinafter indicated as PTPRG or PTPg, is a new specific marker of myeloid or plasmacytoid dendritic cells, both stimulated and unstimulated.

This marker is expressed on the surface of the mammalian DCs and its level of expression is related both/either on the activation and/or differentiation state of the same DCs.

Before the present invention it was unknown that PTPRG is selectively expressed by mammalian myeloid or plasmacytoid DCs.

Today almost 100 PTP are known (Van Huijsduijnen 1998). Molecular cloning studies have revealed the existence of a large PTP superfamily containing enzymes characterized by two or three well conserved tyrosine phosphatase domains. The PTP superfamily consists of classical PTPs, dual specificity (DSPs) and low molecular weight (LMPs) phosphatases. Classical PTPs exist in transmembrane forms (receptor-type PTPs or RPTPs) and non-transmembrane (non-TM) forms and have phosphotyrosine as substrate. RPTPs can be classified in nine subtypes according to the different combinations of the common motifs that compose their external segments. PTPRG forms with RPTPbeta/zeta the subtype V of RPTPs, being characterized by the presence of a carbonic anhydrase-like and a fibronectin type III domain in the N-terminal portion of the extracellular domain (Barnea, Silvennoinen et al. 1993). Hematopoietic cells express a number of RPTPs: CD45 is known to play a role in leukocytes, influencing the lymphocyte signaling process after antigen receptor engagement. Experimental evidence is now emerging concerning the expression and the function of other RPTPs in the hematopoiesis. E.g., CD148 is widely expressed on B and T cells, granulocytes, macrophages, certain dendritic cells and mature thymocytes (de la Fuente-Garcia, Nicolas et al. 1998; Autschbach, Palou et al. 1999). PTPRO is expressed in human stem cells, primary bone marrow megakaryocytes and in human megakaryocytic cell lines (Taniguchi, London et al. 1999), while its alternatively spliced form, PTPROt, is developmentally regulated and implicated in G0/G1 arrest of a specific B cell subpopulation (Aguiar, Yakushijin et al. 1999).

PTPRG was shown to regulate hematopoietic differentiation in a murine embryonic stem cells model (Sorio, Melotti et al. 1997).

In humans, PTPRG mRNA expression is highest in spleen and thymus while in situ hybridization and immunohystochemistry demonstrate a specific expression in irregularly shaped cells identified as dendritic cells by co-expression of lineage-specific markers. In vitro differentiation of monocyte-derived DCs demonstrate that PTPRG expression is increased during the differentiation of monocytes to DCs induced by GM-CSF and IL-4 and is further increased upon maturation.

Macrophages do not express this phosphatase PTPRG both in vitro and in vivo, even when activated by cytokines thus demonstrating a remarkable selectivity of expression within the dendritic cell lineage. PTPg appear to be the only gene to date capable to clearly differentiate DCs from macrophages as confirmed by its selective expression when compared with well established DC-specific genes (DC-LAMP, CCR7, Decysin) that are expressed by activated macrophages.

Since PTPRG is specifically expressed by Dendritic phenotype cells, they allow the subclassification of this cell subset in the hematopoietic system and the identification of the several subsets in which phenotypes are very important to make out the DC biology and their utilization in clinical instruments.

The present invention further relates to methods for selectively identifying myeloid or plasmacytoid DCs either by detecting the level of PTPRG expression by means of one or more antibodies capable of selectively bind the same PTPRG, and therefore capable of identify this specific PTPRG marker, or by measuring both/either PTPRG gene transcription and/or its proteic product expressivity. Moreover, the present invention further relates to the antibodies and the solid phase “chip” with oligonucleotides to perform said method.

As known, reference to “oligonucleotides” should be understood to refer to a nucleotides sequence.

The present invention further relates to a method for evaluating the functional state of the DCs, as well as a method for insulating DCs by a selection process, e.g. a negative process comprising the step of eliminating from a cell population the cells that express the markers not yet expressed by DCs. In another embodiment, the invention relates to a method for identifying a immunomodulating substance as well as for evaluating its in vivo, ex vivo and in vitro immunomodulatory activity.

As known, reference to “ex vivo” should be understood to refer to material after sampling, e.g. form a mammalian.

According to the invention, a method for identifying myeloid or plasmacytoid dendritic cells, both stimulated and unstimulated, e.g. sampled form a mammalian, comprises a first step of preparing the cell sample to handle according to the well-known techniques, a step of placing said cell sample in contact with a compound capable of bind the Receptor type Tyrosine Phosphatase Gamma (PTPRG) of the dendritic cells to form a complex and an another step of detecting said complex.

The compound can be a polypeptide able to selectively bond the PTPRG or a fragment of the same or a oligonucleotide complimentary to mRNA oligonucleotide of the PTPRG to allow the selective detection of the DCs.

The polypeptide is an antibody, or a fragment thereof, developed against a polypeptide constituted by the total sequence, or by a partial sequence of amino acids comprised in the human PTPRG protein sequence and in the Genebank database, access number NM_{—}002841, like an antigen.

Hereinafter, reference to “antibody” should be understood to comprise the complete antibody as well as antibody fragments derived from the same by means of well-known techniques.

In table 2 are indicated preferred antigens. As will appear more clearly in the “MATERIAL and METHODS” section, antibodies developed against P4a, P4b, P5, P6, P7, P8, P9, P15, P16 sequences as antigens are very good markers.

TABLE 2

| | Polypeptide |
|---|---|
| Antibody | Sequences in appendix 1 |
| P1: | CAGPTWQDSKLRRWNFHWAHSNGSAGSEHSINGRRF |
| P2: | CNNFRPQQRL |
| P3: | CRNQTEPSPTPSSPNRT |
| P4a: | CGSDPKRPEMPSKKPMSRGDRFSED |
| P4b: | GSDPKRPEMPSKKPMSRGDRFSEDC |
| | GSDPKRPEMPSKKPMSRGDRFSED |
| P5 | EYL RNN FRP QQR LHD R |
| P6 | NED EKE KTF TKD SDK DLK |
| P7 | CEEG EKD EKS ESE DGE REH |
| P8 | EED GEK DSE KKE KSG VTH |
| P9 | SDP KP EMP SKK PMS RGD R |
| PTM: | CPSSGERGEKGSRK |
| PC: | CKCDQYWP |
| PU: | CQKGNPKGRQN |
| P15 | TVDKNGAVLIADESD |
| P16 | SEDGEREHEEDGEKD |

All sequences have been analyzed with Hopp-Woods e Kyte-Doolittle hydrophilicity algorithm, the Emini exposure probability algorithm, the Karplus-Shulz flexibility algorithm and the Jameson-Wolf algorithm.

Suitably, an effective amount of the antibody, or fragment thereof, is prepared in a composition comprising one or more antibodies with suitable adjuvants and/or excipients and/or solvents, usually used in the art.

Accordingly, the detection of the antigen—antibody complex may be performed applying a technique well known in the art to detect the antigen—antibody complex, e.g. by cytofluorometry, immunofluorescence or immunocyte-histochemistry on cells and/or biological tissues, ELISA essay, immunoprecipitation.

According to the invention, the antibodies, or fragments thereof, may be monoclonal, polyclonal or chimerical antibodies.

It is now well-established in the art that the non-CDR regions of a mammalian antibody may be replaced with similar regions of conspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody.

It is thus possible the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Thus, according to the present invention, reference to “antibody or fragment thereof” should be understood to refer also to F (ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F (ab′) 2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or nonhuman sequences.

The present invention also includes use of so-called single chain antibodies.

All process for providing chimeric antibodies are well known to the persons skilled in the art, and information about it can be found online (e.g: http://users.rcn.com/jkim ball.ma.ultranet/BiologyPages/M/Monoclonals.html) or in literature, such as “Antibody Technology”; E. Liddell e I. Weeks 1995 BIOS Scientific Publishers Taylor & Francis Group plc. 4 Park Square Milton Park Abingdon Oxfordshire OX14 4RN; Antibody Phage Display, Methods and Protocols, O'Brien, Philippa M. (University of Glasgow, Glasgow, UK), Aitken Robert (University of Glasgow, Glasgow, UK), Humana Press, Series: Methods in Molecular Biology, Volume 178, December 2001, Pages: 416, ISBN: 0-89603-711-8.

According to the invention, the compound suitable for identifying dendritic cells in accordance with claim 1 is an oligonucleotide complementary to a PTPRG mRNA oligonucleotide in such a manner as to hybridize and measure the transcription degree of the PTPRG gene both in vivo and ex vivo.

The expression level may be determined by Northern blot, RT-PCR, solid-phase chip.

Standard sequences hybridization technique uses microsequences on solid phase, known as <<microarray>>.

This technique is usually utilized to assess patterns of nucleic acid expression and identify nucleic acid expression, and is disclosed in literature, e.g. in “The Chipping Forecast”, Nature Genetics, vol. 21, January 1999.

Conveniently, a plurality of nucleotides on a solid-phase substrate may be utilized. This substrate, where are located several nucleotide sequences, is usually named “chip”, while the plurality of nucleotides are known as “array”.

Oligonucleotides are a plurality of nucleotide sequences complementary to nucleic acid sequences present in databases of myeloid or plasmacytoid dendritic cells expression and comprise one or more mRNA PTPRG nucleotide sequences.

The step of detecting the complex comprises the comparison of the nucleic acid hybridization model of the cells to be identified with the control expression model into the comparison databases. The expression level of said expression model which is substantially equal to the control levels present into the comparison databases indicates that the sample cells are myeloid or plasmacytoid dendritic cells.

Since the PTPRG is a specific marker of DCs, it is possible to exclude that the detected signal is produced by other cells of the immune system, particularly by macrophages, that are always present in the tissues of the sample.

Cited microsequences technique is also known by other names including DNA chip technology, gene chip technology, and solid-phase nucleic acid array technology. It is based on obtaining an array of identified nucleic acid probes on a fixed substrate, labeling target molecules with reporter molecules, e.g., radioactive, chemiluminescent, or fluorescent tags such as fluorescein, Cy3-dUTP, or Cy5-dUTP, hybridizing target nucleic acids to the probes, and evaluating target-probe hybridization.

A probe with a nucleic acid sequence that perfectly matches the target sequence will, in general, result in detection of a stronger reporter molecule signal than will probes with less perfect matches. Many components and techniques utilized in nucleic acid microarray technology are presented in The Chipping Forecast, Nature.

A solid-phase nucleic acid molecule series (array) consists essentially of a plurality of nucleic acid molecules, expression products thereof, or fragments thereof, wherein at least two and less than all of the nucleic acid molecules selected from the PTPg sequence informations present in public and private databases (including expression products thereof, or fragments thereof) are fixed to a solid substrate. The solid-phase array further comprises at least one control nucleic acid molecule and a number from three to 100 of different nucleic acid molecules, preferably a number of 5 different nucleic acid molecules.

Preferably, microarray substrates may be selected from the group consisting of glass, silica, aluminosilicates, borosilicates, metal oxides such as alumina and nickel oxide, various clays, nitrocellulose, or nylon.

The nucleic acid molecules are fixed to the solid substrate by covalent bonding. Probes are selected from the group of nucleic acids consisting of DNA, genomic DNA, cDNA, oligonucleotides and may be natural or synthetic. Oligonucleotide probes preferably are 20 to 25-mer oligonucleotides and DNA/cDNA probes preferably are 500 to 5000 bases in length, although other lengths may be used. Appropriate probe length may be determined by one of ordinary skill in the art by following art-known procedures. Probes may be purified to remove contaminants using standard methods known to those of ordinary skill in the art such as gel filtration or precipitation.

Probe may be synthesized directly on the substrate, eventually coated with a compound to enhance synthesis such as, e.g., oligoethylene glycols. Coupling agents or groups on the substrate can be used to covalently link the first nucleotide or oligonucleotide to the substrate. These agents contain at least a reactive group selected among amino, hydroxy, bromo, and carboxy groups.

These reactive groups are preferably attached to the substrate through a hydrocarbyl radical such as an alkylene or phenylene divalent radical, one valence position occupied by the chain bonding and the remaining attached to the reactive groups. These hydrocarbyl groups may contain up to about ten carbon atoms, preferably up to about six carbon atoms.

In one embodiment, probes are synthesized directly on the substrate in a predetermined grid pattern using methods such as light-directed chemical synthesis, photochemical deprotection, or delivery of nucleotide precursors to the substrate and subsequent probe production.

In another embodiment, the substrate may be coated with a compound to enhance binding of the probe to the substrate, such as polylysine, amino silanes, amino-reactive silanes (Chipping Forecast, 1999) or chromium (Yasuda, Okano et al. 2000), in a precise, predetermined volume and grid pattern, utilizing a computer-controlled robot to apply probe to the substrate in a contact-printing manner or in a non-contact manner such as ink jet or piezo-electric delivery. Probes may be covalently linked to the substrate with UV-irradiation or heat.

In a further embodiment, the polypetidic compound for identifying dendritic cells in accordance with claim 1 is an array of polypeptide sequences.

In fact, standard techniques of microarray technology may be utilized to assess expression patterns of polypeptides and/or to identify compounds capable of binding said polypeptides. The compounds capable of binding the polypeptides may be compounds such as antibodies, or may be cell constituents (preferably DC constituents) or immunocells (e.g., B and T cells) or the like.

Microarray protein technology, which is also known by other names including protein fragment technology and solid-phase protein sequence technology, is well known to those of ordinary skill in the art and is based on obtaining an array of identified protein polypeptide probes on a fixed substrate, labeling target molecules or biological components of the peptides (MacBeath and Schreiber 2000).

In another embodiment of the invention, antibodies or antigen binding fragments thereof that specifically bind polypeptides selected from the group comprising peptides, polypeptides or fragments thereof derived from the PTPRG sequence are attached to the microarray substrate in accordance with standard attachment methods known in the art, preferably with one or more control peptides or protein molecules attached to the substrate, allowing determination of factors such as peptide or protein quality and binding characteristics, reagent quality and effectiveness, hybridization success, and analysis thresholds and success.

The peptide sequences can comprise either binding partners of the peptides or polypeptides encoded PTPg or, alternatively, can comprise fragments (preferably unique fragments) of the polypeptides or peptides encoded PTPg.

In the first variation, the peptide sequence comprise antibodies or antibody fragments that bind specifically to peptides or polypeptides encoded by the markers listed in the databases.

The peptide sequence analysis can be used alongside of or in place of the nucleic acid array in the methods described herein.

Since MHC class I and II markers are also expressed at high levels in moDC populations either in the resting or stimulated state, the isolation and identification techniques provided herein may include, in addition to the PTPRG, which has not heretofore been identified as selectively present on DCs, other DC-specific markers well known to those with ordinary skill in the art.

E.g., MHC II, CD83 and CD1a receptors are highly expressed on mDC and may be used to select mDC from bulk samples.

A method for identifying a monocytoid dendritic cell may comprise the step of determining the level of expression of at least 5 markers in a test cell, and comparing the level of expression of the at least 5 markers in the test cell with the level of expression in a myeloid cell expression database.

A level of expression of the at least 5 markers in the test cell that is approximately identical to the level of expression of that at least 5 markers in the plasmacytoid expression database indicates that the test cell is a dendritic cell.

A level of expression that is approximately identical to the level of expression in the database is defined as within (i.e., +/−) 20% for measurements of individual markers, preferably within 10%, and even more preferably within 5% of the database expression level for the particular marker.

As an example, an expression level of CD40 in a test cell that is +/20% of the level of expression of CD40 in the 2 hour unstimulated data set is approximately identical to the level of the database. In this latter example, if the level of expression was for example 30%, then this level of expression would not be considered approximately identical, but rather would be characterized as up-regulated relative to the expression level in unstimulated mDC.

Techniques for isolating and purifying dendritic cells from a cell sample in accordance with the present invention comprise a step of identification utilizing one of the methods described herein and a step of isolation or separation, in positive or negative manner, cells that express PTPRG phosphatase based on the degree of expression of the PTPRG alone or based on the expression of a group consisting of several different markers and comprising the PTPRG, determining the level of expression of the antigen—antibody complex or the expression level of the hybridization model of nucleic acids.

Separation may be performed by means of a MACS separation (see Cytometry. 1990; 11(2):231-8. High gradient magnetic cell separation with MACS) or a FACS sorting with suitable cytofluorimeters (Miltenyi S, Muller W, Weichel W, Radbruch A, and http://www.cardiff.ac.uk/medicine/haematology/cytonetuk/introduction_to_fcm/cell_sorting.html).

Accordingly, DCs bonded to the antibody may be separated, either in a positive or negative manner, by means of an immunoprecipation or an affinity chromatography.

An isolated cell is one that is separated from the majority of other different cells with which it is normally in contact in vivo and it can, therefore, be simply handled. An isolated cell is purified when the cell population in which it exists in vitro is greater than 95% pure (i.e., greater than 95% of the cells are the same as the isolated cell (e.g., a moDC, a monocyte or a stromal cell), more preferably greater than 97% pure, and most preferably greater than 99% pure.

The data provided in the databases also allow for the identification and isolation of subsets of cells within the monocytes and mo-DC population. For example, immature and mature moDC can be harvested from the moDC population either prior to in vitro stimulation, or following various times of exposure to an immunostimulatory agent.

Following this operative way it is possible, e.g., isolating a mature subset of moDC from the general moDC population by selecting for cells that express PTPg at higher level that the appropriate controls at the 24 hour LPS stimulation time point. Additionally, mature cells can be selected based on negative selection.

Alternatively, if an immature subset is desired, it is preferable to isolate cells based on those markers expressed and not expressed at the 2 hour unstimulated time point.

As an example, subsets of moDC can be derived by separating cells based on the expression or lack of expression of particular markers, as determined using reagents capable to identify PTPg specifically.

In this way, subsets such as immature and mature moDC can be derived and individually tested for their response to the agents and other stimuli which can be readily tested using the screening methods provided herein.

The expression level of myeloid and plasmacytoid dendritic cells is determined form the detecting level of the antigen-antibody complex or from the expression level of the hybridization model of nucleic acids of the dendritic cells.

A Method for detecting the activity of myeloid or plasmacytoid dendritic cells may comprise a first step of detecting the expression level, a next step of comparing said expression level with the comparison levels expressed by comparison and control cells into the databases. An expression level higher then the expression level of comparison cells indicates an activity of dendritic cells higher then the comparison cells and wherein the expression level of the comparison cells higher then the expression level of dendritic cells indicates an activity of comparison cells higher then dendritic cells.

Nucleic acid molecules include PTPRG gene sequences and, preferably, they correspond to a specific immunological activity.

A method for identifying a candidate agent useful in the modulation of an immune response comprises determining expression of PTPg of nucleic acid molecules in blood, tissues, bone marrow or monocytes, under conditions which, in the absence of a candidate agent, permit a first amount of expression of PTPg. After the step of contacting the isolated or in situ cells with the candidate agent, it is determined the expression of nucleic acid molecules specific for PTPg. An increase or a decrease in expression in the presence of the candidate agent relative to the expression in the absence of the candidate agent indicates that the candidate agent is an immune modulating agent.

Although these markers have been identified following artificial immunostimulation, the findings are applicable to the modulation of any immune response that involves DC, that is for modulating inappropriate immune responses such as autoimmune responses, or uncontrolled and thus detrimental immune responses, such as those to endogenous antigens (autoimmune diseases).

The response may be artificially induced, e.g. in a clinical setting with CpG immunostimulatory nucleic acids or other adjuvants, or may be the result of an infection or an autoimmune disease.

An example of the latter is the in vivo treatment of a mice group with bacterial LPS followed by the sampling of the spleen, that is a known provider of DCs. Because of the preceding allegations, the signal cannot be caused by other immune cells of the tissue, particularly macrophages.

FIG. 9 shows the specific mRNA amplification for PTPRG and for a control gene (G6PDH). Animals treated, although for only 3 h, with LPS shows an increase in the signal relative to PTPRG in comparison with the control.

Immune responses may be modulated either by contacting cells with agents that trigger these markers, or by administering interfering nucleic acids (antisense, micro-RNA, ribozymes, si-RNA) or polypeptides (antennapedia-derived peptides, TAT-containing sequences) or synthesis chemical products that block the translation, induce degradation or reduce/abrogate the function of these markers, as the desired therapeutic effect may be.

With knowledge of a marker that is regulated in response to exposure to immunomodulatory molecules, it is possible to design therapeutic methods for modulating an immune response using antisense nucleic acid molecules specific for such marker, or expression vectors encoding such marker, or compounds which otherwise influence the expression or activity of the marker.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridization with the target gene or transcript.

Those skilled in the art will recognize that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind, selectively with the target under physiological conditions, i.e., to hybridize substantially more to the target sequence than to any other sequence in the target cell under physiological conditions.

In one set of embodiments, the antisense oligonucleotides of the invention may be composed of “natural” deoxyribonucleotides, ribonucleotides, si-RNA or any combination thereof.

That is, the 5′ end of one native nucleotide and the 3′ end of another native nucleotide may be covalently linked, as in natural systems, via a phosphodiester internucleoside linkage. These oligonucleotides may be prepared by art recognized methods which may be carried out manually or by an automated synthesizer. They also may be produced recombinantly by vectors.

In preferred embodiments, however, the antisense oligonucleotides of the invention also may include “modified” oligonucleotides. That is, the oligonucleotides may be modified in a number of ways which do not prevent them from hybridizing to their target but which enhance their stability or targeting or which otherwise enhance their therapeutic effectiveness.

The term “modified oligonucleotide” as used herein describes an oligonucleotide in which (1) at least two of its nucleotides are covalently linked via a synthetic internucleoside linkage (i.e., a linkage other than a phosphodiester linkage between the 5′end of one nucleotide and the 3′ end of another nucleotide) and/or (2) a chemical group not normally associated with nucleic acids has been covalently attached to the oligonucleotide. Preferred synthetic internucleoside linkages are phosphorothioates, alkylphosphonates, phosphorodithioates, phosphate esters, alkylphosphonothioates, phosphoramidates, carbamates, carbonates, phosphate triesters, acetamidates, carboxymethyl esters and peptides.

The term “modified oligonucleotide” also encompasses oligonucleotides with a covalently modified base and/or sugar. For example, modified oligonucleotides include oligonucleotides having backbone sugars which are covalently attached to low molecular weight organic groups other than a hydroxyl group at the 3′position and other than a phosphate group at the 5′position. Thus modified oligonucleotides may include a 2′-O-alkylated ribose group. In addition, modified oligonucleotides may include sugars such as arabinose instead of ribose.

Knowing what markers are expressed by moDC at various times during treatment with a specific compound allows one to tailor a cocktail for further stimulating or potentiating the immune response derived from such a compound.

The immune response that is potentiated is an innate immune response may be a natural or adaptive immune response. May be also provided methods for modulating inflammation and inflammatory processes. For example, the cells can be used to screen for compounds that upregulate (i.e., agonists) or downregulate (i.e., antagonists) inflammatory markers.

The present invention, thus, contemplates pharmaceutical preparations containing modified antisense molecules that are complementary to and hybridizable with, under physiological conditions, nucleic acids corresponding to the marker described, together with pharmaceutically acceptable carriers. Antisense oligonucleotides may be administered as part of a pharmaceutical composition. Such a pharmaceutical composition may include the antisense oligonucleotides in combination with any standard physiologically and/or pharmaceutically acceptable carriers which are known in the art. The compositions should be sterile and contain a therapeutically effective amount of the antisense oligonucleotides in a unit of weight or volume suitable for administration to a patient.

Another unexpected finding of the present invention is the observation that PTPg is expressed, and in some instances at high levels, within 3 hours from the treatment with an immunostimulating agent. Accordingly, since PTPRG expression provides information about nucleic acid molecule expressed by mDC after exposition to this agents, the cells can be used to screen for agents that attenuate or inhibit the expression of PTPg.

A single nucleic acid molecule is a nucleic acid molecule that is capable of uniquely characterizing an immune response.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulated by recombinant DNA techniques well known in the art. Thus, a nucleotide sequence contained in a vector in which 5′ and 3′restriction sites are known or for which polymerase chain reaction (PCR) primer sequences have been disclosed is considered isolated but a nucleic acid sequence existing in its native state in its natural host is not.

An isolated nucleic acid may be substantially purified, but need not be.

For example, a nucleic acid that is isolated within a cloning or expression vector is not pure in that it may comprise only a tiny percentage of the material in the cell in which it resides. Such a nucleic acid is isolated, however, as the term is used herein because it is readily manipulated by, standard techniques known to those of ordinary skill in the art.

Unique fragments can be further used. A “unique fragment” as used herein with respect to a nucleic acid is one that is a “signature” for the larger nucleic acid.

“Agents that increase expression” of a nucleic acid, as used herein, are the sense nucleic acids, polypeptides encoded by the nucleic acids, and other agents that enhance expression of such molecules (e.g., transcription factors specific for the nucleic acids that enhance their expression). Any agents that increase expression of a molecule and capable of increase its activity are useful.

The kinetic analysis of PTPg lends insight into treatment strategies for both enhancing and suppressing an immune response that involve mDC. For example, the expression of a PTPg by a resting mDC indicates that the cell will be responsive to the ligand for that receptor, and further that the cell may be activated by that ligand in the absence of other stimulants such as immunostimulatory bacterial derivatives.

Moreover, expression of PTPg following moDC stimulation (e.g., induced by exposure to bacterial derivatives) indicates that the cell is made responsive to the ligand for that receptor as a result of increased immunostimulation and that its activation state may be heightened by exposure to the ligand.

In yet another example, expression of a PTPg by a stimulated pDC indicates an avenue of immunoregulation of such cells where it is desired to control or suppress an inappropriate immune response (FIGS. 8 and 9).

With respect to this latter embodiment, it may sometimes be desirable to control an induced immune response (e.g., a clinically induced immune response), and this can be achieved by administration of an agent that binds to the negative regulatory marker on the activated mDC.

This molecule, as pointed out by Sorio et al. (Sorio, Mendrola et al. 1995), has tyrosine phosphatase enzymatic activity and may be capable of regulate, either in a positive or negative manner, the immune responses.

Because PTPg is a phosphatase this might work as a negative signalling receptor, methods may be devised to inhibit this killing as well as inhibit general m-DC activation.

A method for determining the effects of immunomodulatory agents, both immunostimulatory or immunoinhibitory, on CDs and for modulating the activity of plasmacytoid or myeloid dendritic cells comprises the steps of: i) identifying the dendritic cells; ii) determining a first activity of said dendritic cells; iii) administering an effective amount of at least an immunomodulatory agent having a receptor on the surface of said cells to modulate the activity of said cells; iv) determining a second activity of said dendritic cells; v) comparing the first and the second activity, wherein a second activity higher then the first indicates that said agent is an immunostimulatory agent and wherein a first activity higher then the second indicates that said agent is an immunoinhibitory agent. A further a step of inducing an immune response by an agent selected from the group consisting of bacterial and/or viral derivative agents, modified or attenuate pathogens, cancer cells or derivates may be provided.

Suitably, the immunomodulatory agent is selected from the group including antibodies, specific antibody fragments for the receptor, ligands for the receptor, polypeptides containing the sequence of PTPg gene in the deduced extracellular domain.

For example, as provided in FIGS. 9 and 11, a population of DC were exposed in vitro to an immunostimulatory agent such as the bacterial lipopolysaccharide (LPS). At 24 hours the cells were harvested and the level of expression of PTPg and other phosphatase or genes reported to DC-specific determined. Thus, the invention further provides information regarding a panel of genes that are induced, suppressed, up-regulated, down-regulated, or unaffected by exposure to immunomodulatory molecules.

This information also allows for moDC to be screened for their ability to respond in vivo to administration of immunostimulatory agents, This latter screening in turn allows for screening of subjects who are most likely to benefit from treatment with such agents.

Although not intending to be bound by any particular theory, it is postulated that the modulation of the PTPRG induced in human cells cultured in vitro and in the mice spleen treated in vivo may roughly approximate the expression within the mDC population in vivo during an injury, infection or disease.

More specifically, the unstimulated time point and its corresponding marker data may be indicative of a mDC in vivo in a subject not having an injury, infection or disease. The 24 hour stimulation time point may be characteristic of a mDC during the time of antigen presentation to other immune cells such as T and B cells in a secondary lymphoid site.

Knowledge of PTPg expression levels leads to the discovery of agents that can be administered at these different functional stages in vivo in order to potentiate or attenuate the ongoing response. Accordingly, administration of immunomodulatory molecules at a particular time post injury (or post active infection in the case of vaccination) and/or at a particular location in a subject (i.e., a secondary lymphoid organ such as the spleen or lymph nodes) may potentiate antigen presentation by such cells.

The PTPRG may be used both for increasing and for controlling the immune responses induced by the stimulation of the DCs.

Advantageously, dendritic cells may be used to design vaccines or specific antigen therapies, such as antitumoral therapies, which that involve priming of immune cells with antigen ex vivo prior to reintroduction into a subject.

Optimal Ag targeting and activation of APCs, especially dendritic cells (DCs), are important in vaccine development. MDCs, which express TLR3, TLR4, and TLR7, responded to poly(I:C), LPS, and imidazoquinolines with phenotypic maturation and high production of IL-12 p70 without producing detectable IFN-alpha.

Optimally TLR ligand-stimulated PDCs or MDCs exposed to CMV or HIV-1 Ags enhanced autologous CMV- and HIV-1-specific memory T cell responses as measured by effector cytokine production compared with TLR ligand-activated monocytes and B cells or unstimulated PDCs and MDCs.

Together, these data show that targeting specific DC subsets using TLR ligands can enhance their ability to activate virus-specific T cells, and modulation of PTPg expression might be a economic, rapid and sensitive readout of the response providing information for the rational design of TLR ligands as adjuvants for vaccines or immune modulating therapies.

The methods for detecting the activity of dendritic cells, for modulating the activity of dendritic cells and for identifying a candidate agent useful in the modulation of an immune response described herein provide, used in a separate, sequential or suitably combined manner, screening techniques or assays useful, e.g., for comparing the ability of other immunostimulatory molecules to induce a PTPg expression similar to or distinct from the expression induced by the immunostimulatory molecules listed.

The present invention allows a rapid and sensitive method to recognize the effects of agents such as immunostimulatory nucleic acid molecules on mDC populations, and allows a more detailed comparison of the effects of such immunomodulatory molecules relative to the effects of immunostimulatory molecules (such as that used in the Examples).

Anyone of ordinary skill in the art may be able to synthesize agents that conformationally mimic immunomodulatory molecules. Such agents or other agents which are conformationally distinct from the one cited in the examples may then be tested using the screening assays of the invention for their ability to similarly mimic the transcriptional effects on PTPg expression on mDC populations.

In case of compounds that mimic part of the immunomodulatory molecules induced response, the presence or the extent of the induction of PTPg expression or its subcellular localization may be exploited as a rapid and cost-effective screening method to identify the active domain of a tested compound.

The screening methods can be further used to determine the effects of other immunostimulatory agents as compared to the effects of LPS. For example, with knowledge of both the biological outcome and the changes in expression pattern induced by LPS treatment, it is now possible to characterize other immunostimulatory agents relative to these two parameters, and to identify and categorize such agents based on their ability to effect the expression levels of PTPg both at the RNA and protein level.

Yet another unexpected finding is the observation that PTPg in stimulated moDCs co-localize with MHCII (FIG. 8).

This phenomenon was not previously known.

As the principal role of mDC consist in the modulation (either in negative or positive manner) of the functional properties of B and T cells, it is possible to derive that a close proximity between these two molecules indicate a cross-talk and a likely important role for PTPg in the regulation of immune response due to its influence on the “immunological synapse” (Krummel and Davis 2002; Trautmann and Valitutti 2003).

This finding is the basis for therapeutic methods for either inducing or attenuating an immune response that involves mDC and optionally T cells, such as the antigen presentation and recognition that occurs between these cell types in secondary lymphoid tissues.

The screening methods described herein for identifying agents that mimic CpG immunostimulatory nucleic acids, can also be used for validating the efficacy of agents. Agents of either known or unknown identity can be analyzed for their effects on PTPg gene expression in moDC using methods such as those described herein.

According to a preferred method, purified populations of moDC or subsets thereof (based on further purification on the basis of a subset of markers from the available databases) are exposed to the agent, preferably in an in vitro culture setting, and after set periods of time, the entire cell population or a fraction thereof is removed and mRNA is harvested therefrom.

Either mRNA or cDNA is then applied to a nucleic acid array such as that used in the Examples or in some embodiments a nucleic acid array that consists of a subset of those markers. Hybridization readouts are then compared to the data of provided herein and conclusions are drawn with respect to the similarity of the action of the agent to that of selected stimuli. These methods can be used for identifying novel agents, including nucleic acid and nucleic acid analog based agents, as well as confirming the identity of agents that are suspected of being immunomodulatory agents.

The expression fingerprints provided herein can also be used as global indicators of dendritic cell stimulation, maturation and immune response efficacy. Dendritic cells grown in vitro, or harvested in a temporal or spatial manner from a subject can be analyzed according to this expression of PTPg in order to more fully characterize the dendritic cell and to determine its potential for immune response involvement, or its past immune response involvement.

The modulated expression of PTPg in moDC provided herein allows one of ordinary skill to determine that set of signalling molecules that are expressed in such cells, thereby allowing a determination of what signaling pathways are activated (and which are not activated) in these cells in different condition chosen according to specific PTPg expression levels chosen by the researcher. Accordingly, if it is desirable to stimulate such cells further, then the cells can be contacted with agents that stimulate a specific pathway known to be active. If on the other hand it is desirable to inhibit the stimulation of such cells, then the cells can be contacted with an agent (s) known to inhibit the same pathway.

In an another aspect, the invention provides a method for characterizing subjects according to their potential ability to respond to an immunomodulatory substance or to determine the effectiveness of a treatment allowed to the subjects.

As used herein, the term “subject” indicate a mammal.

It is actually important to determine whether their mDC are continuing to respond to such treatment, that is ascertaining whether moDC have become unresponsive to the treatment. Efficacy of treatment can be determined by testing for the presence of PTPg mDC in the subject at a certain time or at a certain location in the subject following treatment. Moreover, it is possible to diagnose a disorder, and in particular a stage of the disorder, based on which moDC are present in the subject (and in some instances, preferably at the site of a lesion such as a cancerous lesion) and the expression of PTPg alone or in combination of other markers (such as, for example, CD68, CD1a, CD14, MHC II, CD83).

It has been reported that dendritic cells are reduced in number in the circulation of cancer patients with metastases (Lissoni et al., 1999). Accordingly, a similar analysis of moDC in subjects having a disorder such as cancer, an infectious disease, an allergy, or asthma, can be carried out, by analyzing PTPg transcript or protein expressed in mDC identified in situ or harvested from such subjects and comparing those expression patterns with that of control subjects or of the same subject at different time point during the evolution of the. This type of analysis can provide more fine-tuned, detailed characterization of a disease over and above the classical histological and scant genetic characterizations that are currently available.

Additionally, this type of analysis can be used to flag subjects for less aggressive, more aggressive, and generally more tailored therapy to treat the disorder.

In some subjects, particularly those at risk of developing a disorder which is responsive to dendritic cell therapy, it is possible to stimulate mDC in vivo in order to potentiate antigen specific immune responses, including antigen recognition and uptake by moDC, and antigen presentation by moDC to other immune cells such as T and B cells.

Subjects that are possible candidates for such treatment can be initially screened for the ability of their mDC to respond to such treatment.

In a more specialized application, the mDC can be administered along with an antigen vaccine in order to potentiate the immune response to the vaccine antigen. Preferably the moDC are stimulated either in vitro or in vivo in order to enhance their antigen uptake and presentation functions.

In a further aspect, the invention provides for the use of an effective amount of a immunomodulatory agent as described herein for preparing a drug for modulating the immune response to an infection or to a autoimmune disorder.

In an another embodiment, pharmaceutical compositions for stimulating or unstimulating a the immune response are provided.

These compositions comprise an effective amount of an immunomodulatory agent identified in accordance with the methods described herein with suitable pharmaceutically acceptable excipients or carriers.

The effective amount depend upon the mode of administration, the particular condition being treated and the desired outcome. It will also depend upon the stage of the condition, the age and physical condition of the subject, the nature of concurrent therapy, if any, and like factors well known to the medical practitioner.

In prophylactic applications, the effective amount is a quantity sufficient to delay the onset of, inhibit the progression of, or halt altogether the particular condition being treated, thereby producing patient benefit.

For therapeutic applications, the effective amount is a quantity sufficient to achieve a medically desirable result, thereby producing patient benefit, for example by a reduction in morbidity and/or mortality. In some cases this is a decrease in cell maturation and/or proliferation, or an increase in either of these two parameters.

Generally, the amounts of active compounds in accordance with the present invention are from about 0.01 mg/kg per day to 1000 mg/kg per day.

The methods of the invention, generally speaking, may be practiced using any mode of administration that is medically acceptable, meaning any enteral or parenteral mode that produces effective levels of the active compounds without causing clinically unacceptable adverse effects.

Material and Methods

Antibodies

Antibodies against PTPRG or against polypeptides thereof have been developed by means of techniques well known to the persons skilled in the art and quoted, for example, in “Antibody Technology”; E. Liddell e I. Weeks 1995 BIOS Scientific Publishers Taylor & Francis Group plc. 4 Park Square Milton Park Abingdon Oxfordshire OX14 4RN; in Antibody Phage Display, Methods and Protocols, O'Brien, Philippa M. (University of Glasgow, Glasgow, UK), Aitken Robert (University of Glasgow, Glasgow, UK), Humana Press, Series: Methods in Molecular Biology, Volume 178, December 2001, Pages: 416, ISBN: 0-89603-711-8, either against the complete protein or against polypeptidic sequences of the PTPRG sequence present in Genbank database, accession number NM_{—}002841, or against the extracellular domain and/or polypeptides of the extracellular domain of the PTPRG. In order to be more clear, in appendix 1 is quoted the code sequence, comprising nucleotides from 718 to 50551.

Collection of Normal Hematopoietic Tissues

The Hematology Section provided normal bone marrow and cord blood samples. Peripheral and bone marrow mononuclear cells and polymorphonucleated cells were isolated by centrifugation on density gradient (Lymphoprep-Nycomed Pharma AS, Oslo, Norway) from normal blood and bone marrow aspirates. All the samples have been obtained from informed patients or donors. We analyzed surgical specimens from human tissues including lymphoid organs (bone marrow, thymus, tonsils, reactive lymph nodes and spleen), and non-lymphoid tissues (skin, lung and liver). From each specimen, a portion was snap frozen in isopentane liquid nitrogen and then stored at −80° C.; the remaining tissue block was formalin-fixed paraffin-embedded. Cryostat sections were air dried overnight at room temperature and fixed in acetone for 10′ before staining.

In Situ Hybridization (ISH)

Probe labeling. Segments of DNA corresponding to the desired RNA sequences were amplified from the full-length human PTPg construct (cloned in pCR®3.1, BRL, Milan, Italy) using PCR. Bacteriophage RNA polymerase (T7 and SP6) promoters were incorporated into the termini of the amplified sequences by including the polymerase promoter sequences at the 5′ ends of the PCR primers 20. The T7 and SP6 promoter sequences were linked to the sense and antisense PTPg primers respectively (T7—PTPg:5′ ATT MT ACG ACT CAC TAT AGG GTT TTA CAA TCC AGA TGA CTT TGA 3′; SP6—PTPg [symbol] 5′CGA TTT AGG TGA CAG TAT AGA ATA CCT GTG TAC CGA TM TAG CTG 3′).

Transcription of sense and antisense cRNA was achieved subsequently by using the appropriate RNA polymerase (T7 or SP6 RNA Polymerase, Roche, Milan, Italy). DNA templates were purified using phenol/chloroform extraction and ethanol precipitation and suspended in 10 mM Tris HCl pH 8. To synthesize RNA the following reaction mix was incubated at 37° C. for 2 h: 100-200 ng of purified PCR fragment, 2 μl of 10× concentrated digoxigenin (DIG) RNA labeling mix, 2 μl of 10× concentrated transcription buffer, 2 μl of RNA polymerase (T7 or SP6 RNA Polymerase; all reagents from Roche, Milan, Italy) and diethylpyrocarbonate (DEPC)-treated redistilled water to 20 μl.

Tissue preparation. Fresh frozen human tissues were cut on a cryostat into 10 μm-thick sections and mounted onto poly-L-Lysine slides (Poly-Prep™ slides, Sigma, St. Louis, Mo.). The sections were fixed in 3% paraformaldehyde in 0.1 M Na phosphate buffer, rinsed twice in PBS, once in DEPC-treated water and dehydratated.

ISH protocol. Prehybridization was carried out at 43° C. for 3 h in a buffer containing 50% formamide, 4×SSC, 3×Denhardt's solution, 20 mM Na phosphate buffer pH 6.8, 1% sarcosyl, 250 □g/ml of denatured salmon sperm DNA, 500 μg/ml of yeast tRNA and 20 mM ribonucleoside vanadyl complex (New England BioLabs, Beverly, Mass.). The hybridization was carried out at 43° C. for 12-18 h in the same buffer containing 10% Dextran Sulfate and the antisense DIG labeled probe (30 ng/ml). Controls for specificity were performed on adjacent sections by hybridization with the sense DIG labeled probe. Sections were washed twice at 43° C. in 2×SSC for 15 min and incubated for 30 min at 37° C. in NTE buffer (500 mM NaCl, 10 mM Tris, 1 mM EDTA) containing 20 μg/ml RNase A (Roche, Milan, Italy). The slides were subsequently washed twice at 43° C. for 15 min with 1×SSC and 0.1×SSC, twice with a buffer containing 100 mM Tris HCl, pH 7.5 and 150 mM NaCl and covered for 30 min with the same buffer containing 0.1% Triton X-100 and 2% normal sheep serum (blocking solution).

After decanting the solution sections were incubated for 2 h at 37° C. in a buffer containing 0.1% Triton X-100, 1% normal sheep serum and sheep anti-DIG alkaline phosphatase (Roche, Milan, Italy) diluted 1/294. Slides were then washed twice in blocking solution and incubated 10 min with a buffer containing 100 mM Tris-HCl pH 9.5, 100 mM NaCl, 50 mM MgCl2. Sections were covered with 200 μl color solution (nitroblue tetrazolium NBT, 5-bromo-4-chloro-3-indolyl-phosphate BCIP, levamisole), incubated for 18 h in the dark and subsequently counterstained.

Immunohistochemistry, Double Immunofluorescence and Confocal Microscopy

Serial sections of normal tissues were cut on a cryostat into 10 μm-thick sections, mounted onto poly-L-Lysine slides (Poly-Prep™ slides, Sigma, St. Louis, Mo.), fixed for 5 min at 4° C. in chloroform/acetone (50:50) and air-dried. Slides were post-fixed for 5 min in Zamboni's fixative (2% paraformaldehyde, 15% picric acid, 0.1 M sodium phosphate buffer) and washed 3 times with PBS. To inhibit endogenous phosphatase activity the specimens were incubated for 30 min at room temperature in PBS containing 0.1% phenylhydrazine and washed 3 times with PBS. After a 30 min incubation at room temperature with Protein Blocking Agent (Immunotech, Marseille, France), sections were incubated for 45 min at the same temperature with the primary antibody (PTPg-goat polyclonal IgG, C-18 or PTPz-goat polyclonal IgG, both Santa Cruz Biotechnology, Santa Cruz, Calif.) diluted 1:60. The sections were washed 3 times with PBS and incubated for 30 min at room temperature with a donkey anti-goat IgG biotin-conjugated antibody (Santa Cruz Biotechnology, Santa Cruz, Calif.) diluted 1:250. At the end of 3 additional washes with PBS, sections were incubated for 30 min at room temperature with streptavidin-horseradish peroxidase conjugate (Amersham Pharmacia Biotech, Uppsala, Sweden) diluted 1:200 and washed with water.

Diaminobenzidine was used as peroxidase substrate for 10 min incubation. For double staining, sections were subsequently incubated 45 min at room temperature with CD21 or Cytokeratin 19 (both mouse monoclonal from Santa Cruz Biotechnology, Santa Cruz, Calif.) and visualized with a goat anti-mouse IgG complexed to alkaline phosphatase and a specific substrate.

To identify the nature and phenotype of PTPg positive cells, double immunofluorescence stainings were performed using primary antibodies including CD3 (rabbit; DAKO, Glostrup, Denmark; dilution 1:200), CD4 (SK3; Becton Dickinson, San Jose, Calif., USA; 1:10), CD11c (LeuM5, Becton Dickinson, 1:10), CD20 (L26; DAKO; 1:100), CD21 (1F8, Bio-optica, Milan, 1:20), CD34 (QBEnd/10; NeoMarkers, Fremont, Calif.; 1:30), CD35 (Ber-MAC-DRC, Dako, 1:20), CD68 (KP1; DAKO; 1:100), CD123 (7G3, Pharmingen, San Diego, Calif., USA; 1:40), DC-SIGN (Pharmingen, San Diego, Calif., 1:5), DC-LAMP (104.G4; Immunotech; 1:100), Fattor VIII (Rabbit, Neomarkers, 1:30), Glycoforin (Ret40F, Dako, 1:80) and myeloperoxidase (Rabbit; DAKO; 1:3000), a-Smooth muscle actin (BioGenex, San Ramon, Calif.; 1:100) and Vimentin (V9, BioGenex; 1:100). These primary antibodies were revealed using Texas red-conjugated isotype-specific secondary antibodies; PTPg was revealed using biotin-labelled anti-goat antibody followed by strepatavidin FITC (Vector Laboratories, Inc. Burlingame, Calif., 1:75). Immunostained tissue sections were examined with the fluorescence microscope Olympus BX60.

For confocal analysis, cells were washed in PBS and centrifugated on polylysinated-slides, fixed with 4% paraformaldehyde and permeabilized in PBS containing 0.2% TWEEN-20. The rabbit polyclonal antibody specific for PTPg (anti-synthetic peptide P4 {Sorio, 1995 #74} CGSDPKRPEMPSKKPMSRGDRFSED) was used at 10 mg/ml concentration in 0.1% TWEEN-20-PBS and incubated for 2 hours at R.T. Other monoclonal antibodies FITC conjugated (a-CD83 Immunotech Marseille, France and a-HLA DR BD Biosciences, Milan, Italy) were incubated for 1 h at 4° C. together with secondary Cy3-conjugated goat anti-rabbit antibodies (1:1000 dilution; Amersham, Milan). After final washes in PBS, preparation were mounted on the anti-fading 1,4-diazabicyclo[2,2,2]octane (Sigma) in PBS containing 50% glycerol. All preparations were viewed with a Zaiss LSM 510 confocal microscope equipped with argon (488 nm) and helium/neon (543 nm) excitation beams. Results were analyzed with MetaMorph software (Universal Imaging Corporation, Downingtown, Pa.).

Cell Isolation and Culture

Circulating human monocytes, polymorphonuclear cells and lymphocytes were purified from leucocyte-rich buffy coats obtained from human blood of healthy donors (>95% pure as assessed by morphology) by Percoll (Pharmacia Uppsala, Sweden) gradient centrifugation as described elsewhere 21. Cells were cultured in RPMI 1640 (2×106 cells/well/ml) supplemented with 2 mM glutamine and 10% heat-inactivated FCS, and maintained in a humidified atmosphere with 5% CO2 at 37° C. for various times with or without indicated stimuli. Immature human dendritic cells (iDCs) were obtained in vitro as previously described 22. Briefly purified monocytes were cultured in RPMI 1640 (2×106 cells/well/ml) containing 10% heat-inactivated FCS, 2 mM glutamine and supplemented with 50 ng/ml recombinant human GM-CSF and 20 ng/ml recombinant human IL-4 (Peprotech, Rocky Hill, N.J.) for 5-6 days. To induce maturation, iDCs were treated for 24 h with 100 ng/ml LPS (Escherichia coli serotype 026: B6; Sigma, St. Louis Mo.), or other indicated stimuli.

Reverse Transcription-Polymerase Chain Reaction (RT-PCR)

Total RNA from 5×106 cells/point was prepared using Trizol extraction kit (INVITROGEN, Life Technologies, Rockville, USA) according to the manufacturer's instruction. 1 mg of total RNA was reverse transcribed in a volume of 20 ml with 100 ng of random hexamers primers (Roche, Milan, Italy) and 200 U of SuperScript™ II (BRL, Milan, Italy) at 42° C. for 1 h as described by the manufacturer. Polymerase chain reaction (PCR) was performed in a GeneAmp PCR System 9700 (PE Applied Biosystems, Milan, Italy) for 35 cycles (30 s of denaturation at 94° C., 30 s of annealing at 60° C. and 30 s of elongation at 72° C.), in a volume of 25 ml of reaction buffer containing 0.75 U AmpliTaq (PE Applied Biosystems), 0.4 mM of each primer and 0.2 mM dNTPs (Roche, Milan, Italy). The b-actin mRNA amplification was performed for 22 cycles on the cDNA as positive control of reaction efficiency. The primers used were:

| | |
|---|---|
| bACT F | 5′- GGC ACC CAG CAC AAT GAA G-3′; |
| bACT R | 5′- GCT GAT CCA CAT CTG CTG G-3′; |
| PTPg F958 | 5′- CGT CAC CAG TCT CCT ATT GA-3′; |
| PTPg R 1694 | 5′- GTC TGT CAT GTC GTG GTT CC-3′; |
| CD148 F | 5′- ATG CCA CCG TTT ATT CCC AAG C-3′; |
| CD148 R | 5′- GAC TCG TTA TCG CTG ACT TTC C-3′; |
| CD45 F | 5′- CTT AGG GAC ACG GCT GAC TTC-3′; |
| CD45 R | 5′- GAG TGG TTG TTT CAG AGG CAT TA-3′; |
| PTPe F | 5′- CCG ACA GCA ACG AGA CAA CC-3′; |
| PTPe R | 5′- ATT CCG TTG GGC ATC TTC TTG T-3′; |
| PTPu F | 5′- GCT TGC TGT CCT CAT CCT TCT-3′; |
| PTPu R | 5′- CAC CAT ACG CCA GAA GTC ATA G-3′ |

DC-LAMP and DECYSIN primers are from Vandenabeele S et al 23, CCR7 primers are from Bendriss.

Real-Time Quantitative RT-PCR

cDNA was analysed for the expression of target genes by the SYBR Green I double-stranded DNA binding dye assay, using a PerkinElmer ABI Prism 7000 Sequence Detection System (PE Applied Biosystems). Reactions were denaturated for 10 min at 95° C., and then subjected to 40 two-steps amplification cycles with annealing/extension at 60° C. for 1 min followed by denaturation at 95° C. for 15 s. Values are expressed as arbitrary units relative to b-actin. The primers used were:

| | |
|---|---|
| bACT F | 5′- GGC ACC CAG CAC AAT GAA G-3′; |
| bACT R | 5′- GCT GAT CCA CAT CTG CTG G-3′; |
| PTPg1 F | 5′- GCC TTT ACC GTC ACC CTT ATC-3′; |
| PTPg1 R | 5′- AAA GGT ACT ACT TAT GGG GGC-3′ |

Results

Antibodies

Antibodies developed against peptides of the PTPRG sequence present in Genebank database, accession number NM_{—}0028841, have been selected following solubility criteria in aqueous carriers and antigenicity criteria by means of sequence analysis (si veda: http://www.4adi.com/service/antigen.html).

Antigens for preferred antibodies are indicated in the above mentioned table 2.

Antibody P15 have been described by van Niekerk (van Niekerk and Poels 1999) as a reagent for immunohistochemistry on neoplastic and control tissues.

Antibody P16 has been described by Liu (Liu, Sugimoto et al. 2003) as agent suitable for detecting breast carcinoma cells transfected with antisense compounds.

The capability of antibodies P1, P2, P3, P4a, P4b, PTM, PU, PC P5, P6, P7, P8, P9, P15, P16 to react with specific peptides has been analysed. Although they are all capable of recognizing the correspondent peptide using ELISA technique, it has been surprisingly found that antibodies developed against antigens P4a and P4b are capable of recognizing perfectly cells expressing PTPRG using western blotting, immunoprecipitation, cytofluorimetry and immunohistochemistry.

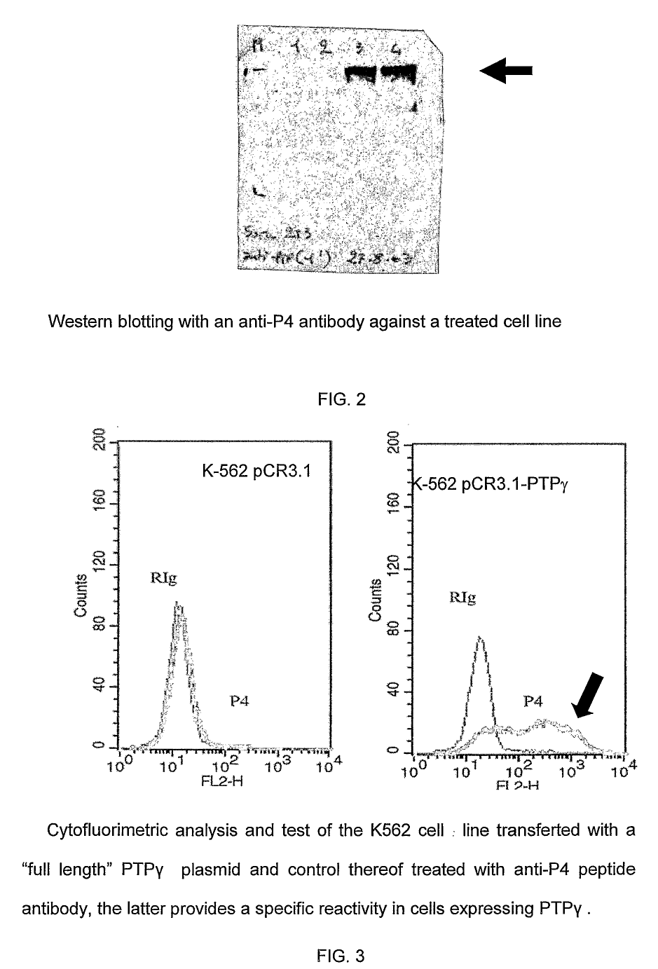

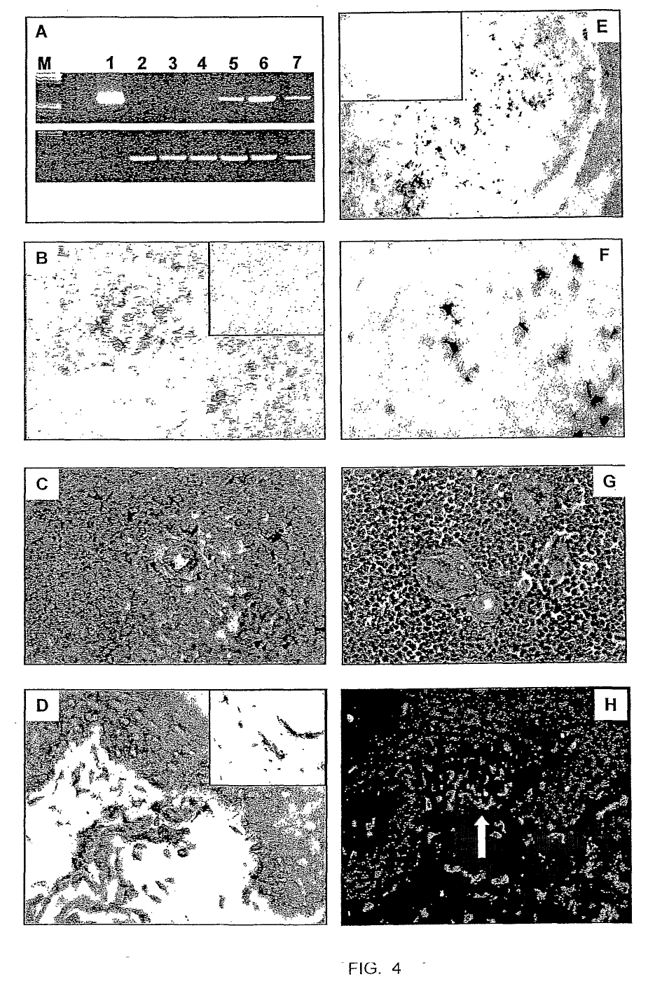

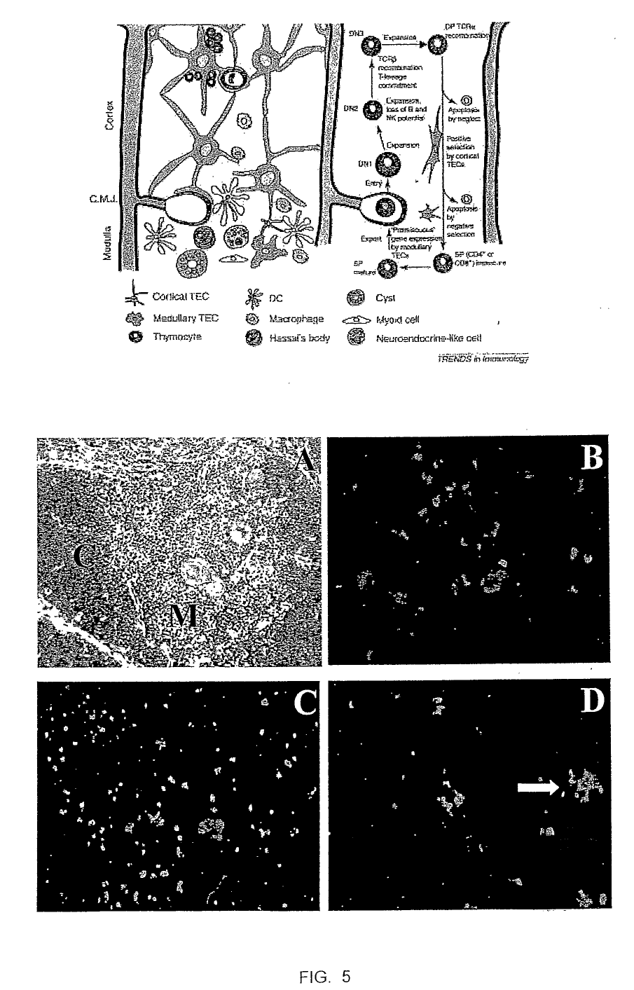

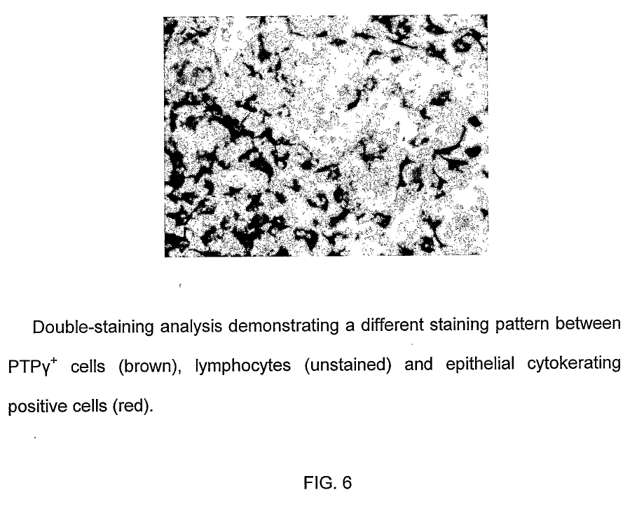

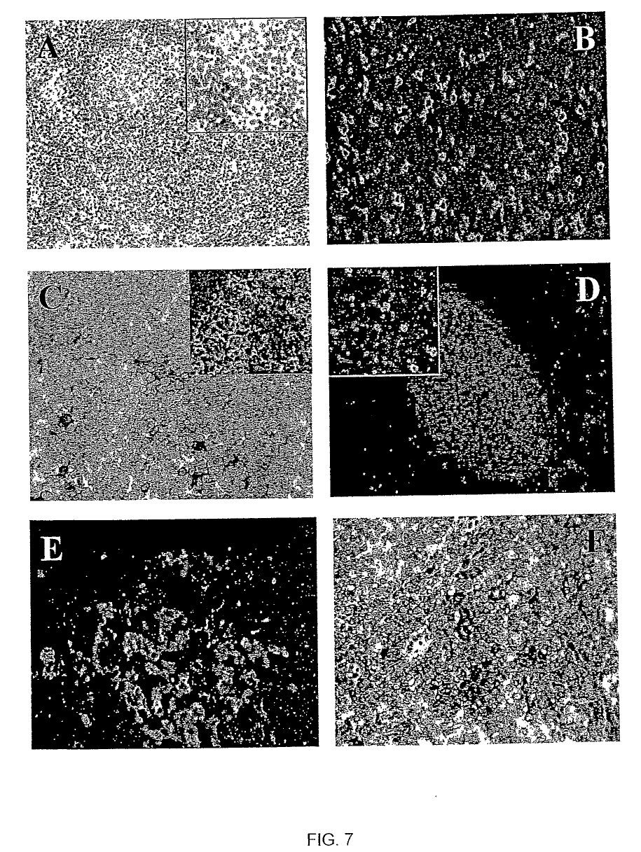

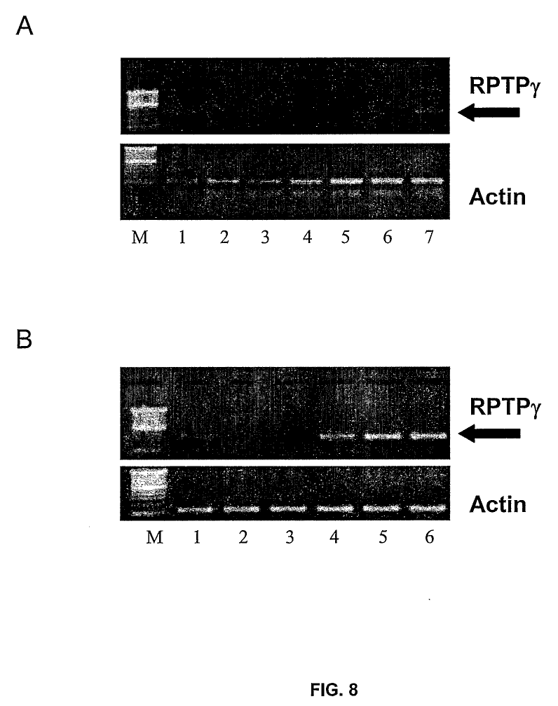

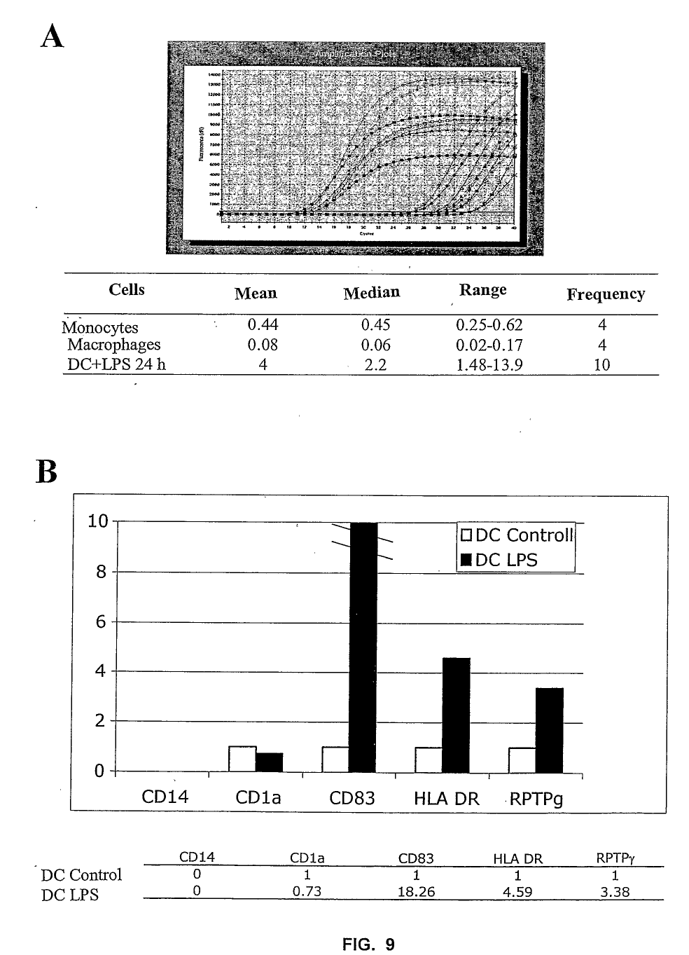

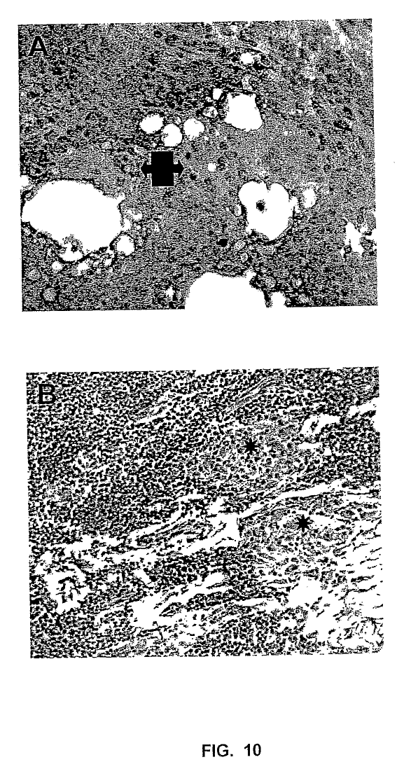

Examples are shown in FIGS. 2 and 3.

PTPg mRNA Expression in Hemopoietic Cells and Tissues

We analyzed PTPg expression in human peripheral blood and organs in which normal, blood cell formation and/or differentiation occurs by a combination of northern blot, RT-PCR, in situ hybridization (ISH) and immunohystochemistry (IHC).

In table 3 are shown the PTPRG expressions in hemolymphopoietic system.

TABLE 3

| Cells/tissues | PTPRG expression | Technique |
|---|---|---|
| Monocytes | + | RT-PCR/IF |
| Limphocytes | − | RT-PCR/Northern blot/RT-PCR |
| PMN | − | IHC/Northern blot/RT-PCR |
| Thimocytes | − | IHC/ISH |
| Limphocytes | − | RT-PCR |
| Th1 and Th2 | | |
| Tissues | − | IHC/ISH |
| macrophages | | |
| MDM | − | IHC/RT-PCR |
| moDC | +(++ after maturation) | IHC/RT-PCR/IF |
| Spleen | Dendritic cells + | IHC/Northern blot/RT-PCR/ISH |
| Thymus | Dendritic cells + | IHC/Northern blot/RT-PCR/ISH |
| Lymphnode | Tingible bodies cells | IHC |
| | (GC) +, sinus lining | |
| | cells + | |
| Tonsil | Tingible bodies cells | IHC |
| | (GC) + | |

PBL: Peripheral Blood Lymphocytes,
MDM: Monocyte-derived macrophages,
MDDC: Monocyte-Derived Dendritic Cells,
PMN: Polimorphonuclear granulocytes,
Th: T lymphocytes, helper phenotype,
GC: Germinal Center

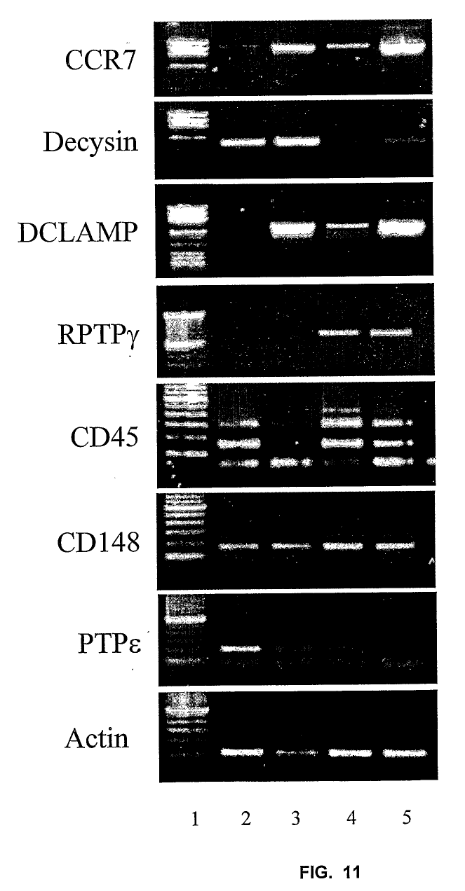

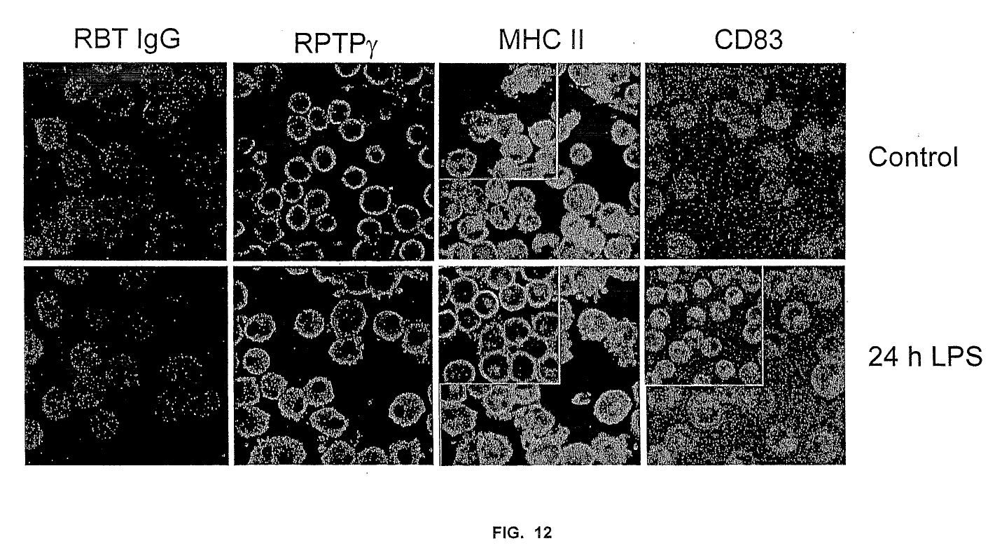

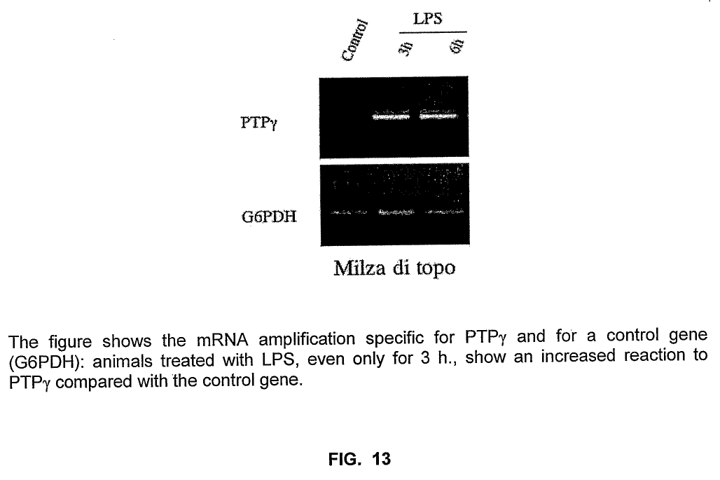

PTPg mRNA expression in peripheral blood cells from normal donors was readibly detectable only in monocytes (Table I, II, FIG. 5), mRNA was readibly detectable in spleen and thymus (FIG. 4A).

The ISH and IHC analysis of three normal spleens revealed the presence of large PTPg positive elements around arterioles and within follicles (germinal center) in the white pulp, while diffuse IHC reactivity was associated with red pulp (FIG. 4, B and C).

The expression of PTPg was analyzed in five normal thymus where epithelial cells form a framework into which progressively differentiating lymphocytes are packed in three areas (the subcapsular zone, cortex and central medulla) and where dendritic cells and macrophages are scattered throughout all regions. ISH allowed the identification of irregularly shaped cells distributed in both medullary and cortical areas (FIG. 4, E-F). Specific antibodies (C-18, Santa Cruz, Calif., USA) confirmed anti PTPg reactivity in the thymus both in the medulla and cortical area (FIGS. 4G and 5A). Dendritic-like cells co-expressing DC-SIGN are displayed in derma too (FIG. 4 D e H).

Positive cells displayed fusate/dendritic morphology but did not react with markers related to the well known thymic DC population including plasmacytoid DC (CD123) (FIG. 5 B) and mature DC (DC-LAMP) (FIG. 5 C). Significantly, as for secondary lymphoid organs, PTPg stained a subset DCSIGN+ (FIG. 5 D) cells, located in the cortex, confirming a preferential expression on monocyte-derived DC.

To completely rule out the epithelial nature of the PTPg positive elements we performed double-staining analyses, shown in FIG. D, that demonstrate a different staining pattern between PTPg positive cells, lymphocytes and cytokeratin positive epithelial cells (not shown). In addition immunohistochemical analysis of 5 thymomas, in which the neoplastic component derives from epithelial cells, further confirmed the lack of reactivity of this population against the same antibody and indicated in the cited figure.

In lymph nodes and tonsils PTPg reactivity was observed in the B-cell compartment, in the interfollicular area and in the nodal marginal sinus. In particular, reactivity within the secondary B-cell follicles was restricted to large non-lymphoid cells (CD20-CD3-) with abundant cytoplasm (FIG. 7A) some of which containing phagocytized debris and thus corresponding to tingible body macrophages (FIG. 7A: insert).

Their identity was further confirmed by double immunofluorescence demonstrating coexpression of CD11c (FIG. 7B). A population of PTPg cells with clear dendritic morphology, was additionally identified in B-cell follicles (FIG. 7C), probably corresponding to the germinal centre dendritic cells.

These PTPg+ cells, indeed, were CD11c+ and CD4+ (FIG. 7C insert); lack of CD21 and CD35 (FIG. 7D) excluded their follicular dendritic cells identity. Double immunofluorescence analysis revealed a preferential clustering of germinal centre T-lymphocytes around PTPg+ cells (FIG. 7D insert) suggesting a role in GC reaction.

Presence of PTPg+ cells was not limited to B-cell compartment, since this protein was expressed on DCSIGN+ sinus macrophages (FIG. 7E) and DCSIGN+ cells in the interfollicular area (FIG. 7F) (data not shown, Vermi, 2003, personal communication). The latter population clearly differentiate from conventional Langerhans-derived interdigitating DC of the paracortex, since they lack CD1a and Langerin, and displayed an immature phenotype (Vermi, Bonecchi et al. 2003). Absence of PTPg was observed in other DC in lymphoid organs such as interdigitating dendritic cells of the T-cell area and plasmacytoid dendritic cells (data not shown).

Time-Course of PTPg Expression in Peripheral Blood Monocytes and Modulation by Cytokines.

Since myeloid DCs derive from monocytes, it has been analysed the presence of PTPRG and the time course. Upon plating, monocytes down-modulate PTPg expression in a few hours (FIG. 8, panel A), while the presence of IL-4 and the combination of IL-4 and GM-CSF maintain the levels of expression for the five days necessary to obtain immature DCs from the latter treatment, that express higher transcript levels (FIGS. 8 A and B). When the cells are cultured in the presence of GM-CSF and IL-4 is added after two days of culture PTPg transcript is re-expressed (FIG. 8 B).

This induction does not occur if IL-4 is added after the complete differentiation of monocytes to macrophages. (data not shown).

PTPg is Specifically Expressed Along the Dc Differentiation Pathway.

Since it has been identified tissue mo-DCs and some cells considered specialized macrophages as strong PTPg expressors, it has been wondered if in vitro cultured macrophages express this gene and if we could induce expression in vitro under specific culture conditions.

Macrophages obtained in vitro by culturing circulating monocytes failed to express PTPg both in resting condition and after treatment with the following cytokines: IL-1b, TNFa, IL-4, GM-CSF, IL-4+GM-CSF, IFNg, IL-10 and with activating molecules (CD40L, LPS) (data not shown). Furthermore, since the PTPg-positive “tingible bodies” macrophages of the germinal center exert phagocytic activity (Baumann, Kolowos et al. 2002), it has been tested the hypothesis that phagocytosis could trigger PTPg expression in in vitro cultured macrophages.

However, induction of phagocytosis by yeast-IgG did not lead to PTPg expression (data not shown).

It has been quantified the levels of PTPRG induction both as protein and transcript in several cell population derivates from monocyties of peripheral blood. Accordingly, in vitro differentiated DCs are utilised as reference and compared with the expression level of monocytes, macrophages and activated DCs.

When monocytes from the same donors were differentiated in the presence of IL-4 and GM-CSF, PTPg expression was kept and resulted to be induced around 2 times at the end of the treatment (FIG. 9A). Addition of well known DC differentiation-inducing agents (bacterial lipopolisaccaride: LPS) lead to a strong and reproducible increase in PTPg mRNA and protein expression, as judged by semi-quantitative RT-PCR (FIG. 8), PCR-Real Time (FIG. 9A) and by the analysis of the intensity of the fluorescence associated to the specific antibodies CD14, CD1a, CD83, HLA DR, PTPRG (FIG. 9B). In the panel A of the same figure is displayed the QRTPCR analysis of the relative levels of RPTPg transcript in myeloid cells from peripheral blood and in DCs treated with LPS relative to expression in Resting DCs (=1). DC=monocytes cultured in the presence of GM-CSF and IL-4 for 5 days, Macrophages=Monocytes cultured complete medium without cytokine addition for 6 days.

Upper picture shows the amplification curves of the fluorescence signal associate to the cDNA quantity of target genes generated after 40 PCR operations. It is possible thanks to the SYBR Green I, a fluorescent molecule that fits the cDNA double helix proportionally to the latter. Data obtained have been first normalised according to the detected quantity of an house-keeping gene (beta-actine), and then according to the target gene in resting conditions (resting DC).

In the panel B of the same figure are graphically shown the data displaying the intensity of the fluorescence of the specific bond of antibodies against antigens indicated (CD14, CD1a, CD83, HLA-DR e PTPRG) in relation to immature DCs (value=1). The value 0 means that no marker expression has been detected.

TNFa and CD40L were also capable of inducing PTPg expression (data not shown).

Moreover, data obtained show the way in which the in vitro macrophages express very few levels of PTPRG specific transcript (FIG. 9A).

Activated Macrophages do not Express PTPg In Vivo.

In order to confirm the lack of expression in macrophages in vivo, pathologic conditions known to involve these cells have been analysed: tissue section of lymph nodes from patients affected by different types of lymphadenitis were stained with a specific antibody. Absence of reactivity was observed on multinucleated giant cells in a case of foreing body reaction as well as in epitheliod and cells from Langhans-like granulomatous lymphadenitis due to mycobacterial infections (FIG. 10 A, B).

This confirm the significance of the in vitro results and indicate that activation of monocyte-derived macrophages in the context of infectious and non-infectious stimuli does not require up-regulation of PTPg.

Other Receptor Tyrosine Phosphatases are Differentially Regulated.

In order to determine the specificity of PTPg modulation, the modulation of the expression profile of three receptorial PTPs known to be expressed by hemopoietic cells has been measured. CD45, CD148 and PTPe are all expressed by in vitro differentiated macrophages and are down-modulated upon LPS treatment. In DCs the same genes are equally expressed and down-modulated upon LPS treatment as in macrophages. Conversely, PTPg is not expressed by macrophages both in resting and after LPS-treatment, it is expressed by DCs but, at variance with all the other phosphatases analyzed, PTPg is up-modulated by LPS treatment (FIG. 11).

PTPg is a Specific Marker of Dendritic Cells

It has been next wondered if PTPg could be regarded as a DC-specific marker in comparison with other genes considered selectively of preferentially expressed by dendritic cells. It has been investigated the expression of DC-LAMP 28, Decysin 29 and CCR730 in the same cell population analyzed for receptor tyrosine phosphatases. It could confirm the expression of these genes in DCs in resting (DC-LAMP and CCR7) and activated conditions (all three). However, while it is possible to consider the selected genes as specific markers in resting conditions, LPS-treated macrophages expressed high levels of all the selected transcripts (FIG. 11). This, again, is at variance with PTPg transcript which is not detected in macrophages in all the experimental conditions tested.

PTPg Co-Localize with MHC II on DCs Plasmamembrane

Confocal microscopy analysis have been performed in order to confirm the surface expression of this receptor-like molecule in DCs. Moreover, since it is well known that DCs maturation induced by stimuli like LPS are associated to the increase of MHC II and CD83 expression on the cell surface, it has been wondered if PTPRG could co-localize.

These analysis show as the phosphatase signal overlaps the MCH II signal (HLA-DR) in activated conditions. The specificity of the result was confirmed by the observation that CD83, another cell surface molecule strongly induced by the same treatment on the cell membrane of the same cellular population, does not appear to co-localize with PTPg (FIG. 12).

### APPENDIX 1

```
PTPRG sequences, selection: 718-5055
  1: M R R L L E P C W W I L F L K I T S S V
ATGCGGAGGTTACTGGAACCGTGTTGGTGGATTTTGTTCCTGAAAATCACCAGTTCCGTG
      1 ---------+---------+---------+---------+---------+---------+
  1: L H Y V V C F P A L T E G Y V G A L H E
CTCCATTATGTCGTGTGCTTCCCCGCGTTGACAGAAGGCTACGTTGGGGCCCTGCACGG
    61 ---------+---------+---------+---------+---------+---------+
  1: N R H G S A V Q I R R R K A S G D P Y W
AATAGACACGGCAGCGCAGTGCAGATCCGCAGGCGCAAGGCTTCAGGCGACCCGTACTG
  121 ---------+---------+---------+---------+---------+---------+
  1: A Y S G A Y G P E H W V T S S V S C G S
GCCTACTCTGGTGCCTATGGTCCTGAGCACTGGGTCACGTCTAGTGTCAGCTGTGGGAC
  181 ---------+---------+---------+---------+---------+---------+
1: R H Q S P I D I L D Q Y A R V G E E Y Q
CGTCACCAGTCTCCTATTGACATTTTAGACCAGTATGCGCGTGTTGGGGAAGAATACCAG
  241 ---------+---------+---------+---------+---------+---------+
  1: E L Q L D G F D N E S S N K T W M K N T
GAACTGCAACTCGATGGCTTCGACAATGAGTCTTCTAACAAAACCTGGATGAAAAACACA
  301 ---------+---------+---------+---------+---------+---------+
                                      (P1)C A G P T W
  1: G K T V A I L L K D D Y F V S G A G L P
GGGAAAACAGTCGCCATCCTTCTGAAAGACGACTATTTTGTCAGTGGAGCTGGTCTACCT
  361 ---------+---------+---------+---------+---------+---------+
        Q D S K L R R W N F H W A H S N G S A G
  1: G R F K A E K V E F H W G H S N G S A G
GGCAGATTCAAAGCTGAGAAGGTGGAATTTCACTGGGGCCACAGCAATGGCTCAGCGGC
  421 ---------+---------+---------+---------+---------+---------+
        S E H S I N G R R F
  1: S E H S I N G R R F P V E M Q I F F Y N
TCTGAACACAGCATCAATGGCAGGAGGTTTCCTGTTGAGATGCAGATTTTCTTTTACAAT
  481 ---------+---------+---------+---------+---------+---------+
  1: P D D F D S F Q T A I S E N R I I G A M
CCAGATGACTTTGACAGCTTTCAAACCGCAATTTCTGAGAACAGAATAATCGGAGCCATG
  541 ---------+---------+---------+---------+---------+---------+
  1: A I F F Q V S P R D N S A L D P I I H G
GCCATATTTTTTCAAGTCAGTCCGAGGGACAATTCTGCACTGGATCCTATTATCCACGGG
  601 ---------+---------+---------+---------+---------+---------+
  1: L K G V V H H E K E T F L D P F V L R D
TTGAAGGGTGTCGTACATCATGAGAAGGAGACCTTTCTGGATCCTTTCGTCCTCCGGGAC
  661 ---------+---------+---------+---------+---------+---------+
  1: L L P A S L G S Y Y R Y T G S L T T P P
CTCCTGCCTGCATCCCTGGGCAGCTATTATCGGTACACAGGTTCCTTGACCACACCACCG
  721 ---------+---------+---------+---------+---------+---------+
  1: C S E I V E W I V F R R P V P I S Y H Q
TGTAGCGAAATAGTGGAGTGGATAGTCTTCCGGAGACCCGTCCCCATCTCTTACCATCAG
  781 ---------+---------+---------+---------+---------+---------+
  1: L E A F Y S I F T T E Q Q D H V K S V E
CTTGAGGCTTTTTATTCCATCTTCACCACGGAGCAGCAAGACCATGTCAAGTCGGTGGAG
  841 ---------+---------+---------+---------+---------+---------+
        (P2)C N N F R P Q Q R L
  1: Y L R N N F R P Q Q R L H D R V V S K S
TATCTGAGAAATAACTTTCGACCACAGCAGCGTCTGCATGACAGGGTGGTGTCCAAGTCC
  901 ---------+---------+---------+---------+---------+---------+
  1: A V R D S W N H D M T D F L E N P L G T
GCCGTCCGTGACTCCTGGAACCACGACATGACAGACTTCTTAGAAAACCCACTGGGGACA
  961 ---------+---------+---------+---------+---------+---------+
  1: E A S K V C S S P P I H M K V Q P L N Q
GAAGCCTCTAAAGTTTGCAGCTCTCCACCCATCCACATGAAGGTGCAGCCTCTGAACCAG
1021 ---------+---------+---------+---------+---------+---------+
  1: T A L Q V S W S Q P E T I Y H P P I M N
ACGGCACTGCAGGTGTCCTGGAGCCAGCCGGAGACTATCTACCACCCACCCATCATGAC
1081 ---------+---------+---------+---------+---------+---------+
  1: Y M I S Y S W T K N E D E K E K T F T K
TACATGATCTCCTACAGCTGGACCAAGAATGAGGACGAGAAGGAGAAGACGTTTACAAAG
1141 ---------+---------+---------+---------+---------+---------+
  1: D S D K D L K A T I S H V S P D S L Y L
GACAGCGACAAAGACTTGAAAGCCACCATTAGCCATGTCTCACCCGATAGCCTTTACCTG
1201 ---------+---------+---------+---------+---------+---------+
  1: F R V Q A V C R N D M R S D F S Q T M L
TTCCGAGTCCAGGCCGTGTGTCGGAACGACATGCGCAGCGACTTTAGCCAGACGATGCG
1261 ---------+---------+---------+---------+---------+---------+
  1: F Q A N T T R I F Q G T R I V K T G V P
TTTCAAGCTAATACCACTCGAATATTCCAAGGGACCAGAATAGTGAAAACAGGAGTGCCC
1321 ---------+---------+---------+---------+---------+---------+
1: T A S P A S S A D M A P I S S G S S T W
ACAGCGTCTCCTGCCTCTTCAGCCGACATGGCCCCCATCAGCTCGGGGTCTTCTACCTGG
1381 ---------+---------+---------+---------+---------+---------+
  1: T S S G I P F S F V S M A T G M G P S S
ACGTCCTCTGGCATCCCATTCTCATTTGTTTCCATGGCAACTGGGATGGGCCCCTCCTCC
1441 ---------+---------+---------+---------+---------+---------+
  1: S G S Q A T V A S V V T S T L L A G L G
AGTGGCAGCCAGGCCACAGTGGCCTCGGTGGTCACCAGCACGCTGCTCGCCGGCCTGG
1501 ---------+---------+---------+---------+---------+---------+
  1: F G G G G I S S F P S T V W P T R L P T
TTCGGCGGTGGTGGCATCTCCTCTTTCCCCAGCACTGTGTGGCCCACGCGCCTCCCGAG
1561 ---------+---------+---------+---------+---------+---------+
  1: A A S A S K Q A A R P V L A T T E A L A
GCCGCCTCAGCCAGCAAGCAGGCGGCTAGGCCAGTCCTAGCCACCACAGAGGCCTTGCT
1621 ---------+---------+---------+---------+---------+---------+
  1: S P G P D G D S S P T K D G E G T E E G
TCTCCAGGGCCCGATGGTGATTCGTCACCAACCAAGGACGGCGAGGGCACCGAGGAAGA
1681 ---------+---------+---------+---------+---------+---------+
  1: E K D E K S E S E D G E R E H E E D G E
GAGAAGGATGAGAAAAGCGAGAGTGAGGATGGGGAGCGGGAGCACGAGGAGGATGGAG
1741 ---------+---------+---------+---------+---------+---------+
                                              (P3)C R N Q
  1: K D S E K K E K S G V T H A A E E R N Q
AAGGACTCCGAAAAGAAGGAGAAGAGTGGGGTGACCCACGCTGCCGAGGAGCGGAATCG
1801 ---------+---------+---------+---------+---------+---------+
        T E P S P T P S S P N R T
  1: T E P S P T P S S P N R T A E G G H Q T
ACGGAGCCCAGCCCCACACCCTCGTCTCCTAACAGGACTGCCGAGGGAGGGCATCAGAT
1861 ---------+---------+---------+---------+---------+---------+
  1: I P G H E Q D H T A V P T D Q T G G R R
ATACCTGGGCATGAGCAGGATCACACTGCCGTCCCCACAGACCAGACGGGCGGAAGGAG
1921 ---------+---------+---------+---------+---------+---------+
  1: D A G P G L D P D M V T S T Q V P P T A
GATGCCGGCCCAGGCCTGGACCCCGACATGGTCACCTCCACCCAAGTGCCCCCCACCGC
1981 ---------+---------+---------+---------+---------+---------+
                  (P4a)C G S D P K R P E M P S K K P
  1: T E E Q Y A G S D P K R P E M P S K K P
                      (P4b)G S D P K R P E M P S K K P
ACAGAGGAGCAGTATGCAGGGAGTGATCCCAAGAGGCCCGAAATGCCATCTAAAAAGCCT
2041 ---------+---------+---------+---------+---------+---------+
        M S R G D R F S E D
  1: M S R G D R F S E D S R F I T V N P A E
        M S R G D R F S E D C
ATGTCCCGCGGGGACCGATTTTCTGAAGACAGCAGATTTATCACTGTTAATCCAGCGGAA
2101 ---------+---------+---------+---------+---------+---------+
  1: K N T S G M I S R P A P G R M E W I I P
AAAAACACCTCTGGAATGATAAGCCGCCCTGCTCCAGGGAGGATGGAGTGGATCATCCCT
2161 ---------+---------+---------+---------+---------+---------+
  1: L I V V S A L T F V C L I L L I A V L V
CTGATTGTGGTATCAGCCTTGACCTTCGTGTGCCTCATCCTTCTCATTGCTGTGCTCGTT
2221 ---------+---------+---------+---------+---------+---------+
  1: Y W R G C N K I K S K G F P R R F R E V
TACTGGAGAGGGTGTAACAAAATAAAGTCCAAGGGCTTTCCCAGACGTTTCCGTGAAGTG
2281 ---------+---------+---------+---------+---------+---------+
(PTM)C P S S G E R G E K G S R K
  1: P S S G E R G E K G S R K C F Q T A H F
CCTTCTTCTGGGGAGAGAGGAGAGAAGGGGAGCAGAAAATGTTTTCAGACTGCTCATTTC
2341 ---------+---------+---------+---------+---------+---------+
  1: Y V E D S S S P R V V P N E S I P I I P
TATGTGGAAGACAGCAGTTCACCTCGAGTGGTCCCTAATGAAAGTATTCCTATTATTCCT
2401 ---------+---------+---------+---------+---------+---------+
  1: I P D D M E A I P V K Q F V K H I G E L
ATTCCGGATGACATGGAAGCCATTCCTGTCAAACAGTTTGTCAAACACATCGGTGAGCTC
2461 ---------+---------+---------+---------+---------+---------+
  1: Y S N N Q H G F S E D F E E V Q R C T A
TATTCTAATAACCAGCATGGGTTCTCTGAGGATTTTGAGGAAGTCCAGCGCTGTACTGCT
2521 ---------+---------+---------+---------+---------+---------+
  1: D M N I T A E H S N H P E N K H K N R Y
GATATGAACATCACTGCAGAGCATTCCAATCATCCAGAAAACAAGCACAAAAACAGATAC
2581 ---------+---------+---------+---------+---------+---------+
  1: I N I L A Y D H S R V K L R P L P G K D
ATCAACATTTTAGCATATGATCACAGTAGGGTGAAGTTAAGACCTTTACCAGGAAAAGAC
2641 ---------+---------+---------+---------+---------+---------+
  1: S K H S D Y I N A N Y V D G Y N K A K A
TCTAAGCACAGCGACTACATTAATGCAAACTATGTTGATGGTTACAACAAAGCAAAAGCC
2701 ---------+---------+---------+---------+---------+---------+
  1: Y I A T Q G P L K S T F E D F W R M I W
TACATTGCCACCCAAGGACCTTTGAAGTCTACATTTGAAGATTTCTGGAGGATGATTTGG
2761 ---------+---------+---------+---------+---------+---------+
                                                        (PC)C K
  1: E Q N T G I I V M I T N L V E K G R R K
GAACAAAACACTGGAATCATTGTGATGATTACGAACCTTGTGGAAAAAGGAAGACGAAAA
2821 ---------+---------+---------+---------+---------+---------+
        C D Q Y W P
  1: C D Q Y W P T E N S E E Y G N I I V T L
TGTGATCAGTATTGGCCAACAGAGAACAGTGAGGAATATGGAAACATTATTGTCACGCTG
2881 ---------+---------+---------+---------+---------+---------+
  1: K S T K I H A C Y T V R R F S I R N T K
AAGAGCACAAAAATACATGCCTGCTACACTGTTCGTCGTTTTTCAATCAGAAATACAAAA
2941 ---------+---------+---------+---------+---------+---------+
            (PU)C Q K G N P K G R Q N
  1: V K K G Q K G N P K G R Q N E R V V I Q
GTGAAAAAGGGTCAGAAGGGAAATCCCAAGGGTCGTCAGAATGAAAGGGTAGTGATCCG
3001 ---------+---------+---------+---------+---------+---------+
  1: Y H Y T Q W P D M G V P E Y A L P V L T
TATCACTATACACAGTGGCCTGACATGGGAGTTCCCGAGTATGCCCTTCCAGTACTGACT
3061 ---------+---------+---------+---------+---------+---------+
  1: F V R R S S A A R M P E T G P V L V H C
TTCGTGAGGAGATCCTCAGCAGCTCGGATGCCAGAAACGGGCCCTGTGTTGGTGCACTC
3121 ---------+---------+---------+---------+---------+---------+
  1: S A G V G R T G T Y I V I D S M L Q Q I
AGTGCTGGTGTGGGCAGAACAGGCACCTATATTGTAATAGACAGCATGCTGCAACAGATA
3181 ---------+---------+---------+---------+---------+---------+
  1: K D K S T V N V L G F L K H I R T Q R N
AAAGACAAAAGCACAGTTAACGTCCTGGGATTCCTGAAGCATATCAGGACACAGCGTAAC
3241 ---------+---------+---------+---------+---------+---------+
  1: Y L V Q T E E Q Y I F I H D A L L E A I
TACCTCGTCCAGACTGAGGAGCAGTACATTTTCATCCATGATGCCTTGTTGGAAGCCATT
3301 ---------+---------+---------+---------+---------+---------+
  1: L G K E T E V S S N Q L H S Y V N S I L
CTTGGAAAGGAGACTGAAGTATCTTCAAATCAGCTGCACAGCTATGTTAACAGCATCCTT
3361 ---------+---------+---------+---------+---------+---------+
  1: I P G V G G K T R L E K Q F K L V T Q C
ATACCAGGAGTAGGAGGAAAGACACGACTGGAAAAGCAATTCAAGCTGGTCACACAGTGT
3421 ---------+---------+---------+---------+---------+---------+
  1: N A K Y V E C F S A Q K E C N K E K N R
AATGCAAAATATGTGGAATGTTTCAGTGCTCAGAAAGAGTGTAACAAAGAAAAGAACAGA
3481 ---------+---------+---------+---------+---------+---------+
  1: N S S V V P S E R A R V G L A P L P G M
AACTCTTCAGTTGTGCCATCTGAGCGTGCTCGAGTGGGTCTTGCACCATTGCCTGGAATG
3541 ---------+---------+---------+---------+---------+---------+
  1: K G T D Y I N A S Y I M G Y Y R S N E F
AAAGGAACAGATTACATTAATGCTTCTTATATCATGGGCTATTATAGGAGCAATGAATTT
3601 ---------+---------+---------+---------+---------+---------+
  1: I I T Q H P L P H T T K D F W R M I W D
ATTATAACTCAGCATCCTCTGCCACATACTACGAAAGATTTCTGGCGAATGATTTGGGAT
3661 ---------+---------+---------+---------+---------+---------+
  1: H N A Q I I V M L P D N Q S L A E D E F
CATAACGCACAGATCATTGTCATGCTGCCAGACAACCAGAGCTTGGCAGAAGATGAGTTT
3721 ---------+---------+---------+---------+---------+---------+
  1: V Y W P S R E E S M N C E A F T V T L I
GTGTACTGGCCAAGTCGAGAAGAATCCATGAACTGTGAGGCCTTTACCGTCACCCTTATC
3781 ---------+---------+---------+---------+---------+---------+
  1: S K D R L C L S N E E Q I I I H D F I L
AGCAAAGACAGACTGTGCCTCTCTAATGAAGAACAAATTATCATCCATGACTTTATCCTT
3841 ---------+---------+---------+---------+---------+---------+
  1: E A T Q D D Y V L E V R H F Q C P K W P
GAAGCTACACAGGATGACTATGTCTTAGAAGTTCGGCACTTTCAGTGTCCCAAATGGCCT
3901 ---------+---------+---------+---------+---------+---------+
  1: N P D A P I S S T F E L I N V I K E E A
AACCCAGATGCCCCCATAAGTAGTACCTTTGAACTTATCAACGTCATCAAGGAAGAGGCC
3961 ---------+---------+---------+---------+---------+---------+
  1: L T R D G P T I V H D E Y G A V S A G M
TTAACAAGGGATGGTCCCACCATTGTTCATGATGAGTATGGAGCAGTTTCAGCAGGAATG
4021 ---------+---------+---------+---------+---------+---------+
  1: L C A L T T L S Q Q L E N E N A V D V F
TTATGTGCCCTTACCACCCTGTCCCAGCAACTGGAGAATGAAAATGCTGTGGATGTTTTC
4081 ---------+---------+---------+---------+---------+---------+
  1: Q V A K M I N L M R P G V F T D I E Q Y
CAGGTTGCAAAAATGATCAATCTTATGAGGCCTGGAGTATTCACAGACATTGAACAATAC
4141 ---------+---------+---------+---------+---------+---------+
  1: Q F I Y K A R L S L V S T K E N G N G P
CAGTTCATCTATAAAGCAAGGCTTAGCTTGGTCAGCACTAAAGAAAATGGAAATGGTCCC
4201 ---------+---------+---------+---------+---------+---------+
  (P15)T V D K N G A V L I A D E S D
  1: M T V D K N G A V L I A D E S D P A E S
ATGACAGTAGACAAAAATGGTGCTGTTCTTATTGCAGATGAATCAGACCCTGCTGAGAGC
4261 ---------+---------+---------+---------+---------+---------+
  1: M E S L V *
ATGGAGTCCCTAGTGTGA
4321 ---------+---------
```

### REFERENCES

Adelman, J. P., J. S. Hayflick, et al. (1983). “In vitro deletional mutagenesis for bacterial production of the 20,000-dalton form of human pituitary growth hormone.” DNA 2(3): 183-93.
Aguiar, R. C., Y. Yakushijin, et al. (1999). “PTPROt: an alternatively spliced and developmentally regulated B-lymphoid phosphatase that promotes G0/G1 arrest.” Blood 94(7): 2403-13.
Ardavin, C. (1997). “Thymic dendritic cells.” Immunol Today 18(7): 350-61.
Autschbach, F, E. Palou, et al. (1999). “Expression of the membrane protein tyrosine phosphatase CD148 in human tissues.” Tissue Antigens 54(5): 485-98.
Balfour, B. M., H. A. Drexhage, et al. (1981). “Antigen-presenting cells, including Langerhans cells, veiled cells and interdigitating cells.” Ciba Found Symp 84: 281-301.
Banchereau, J. and R. M. Steinman (1998). “Dendritic cells and the control of immunity.” Nature 392(6673): 245-52.
Barnea, G., O. Silvennoinen, et al. (1993), “Identification of a carbonic anhydrase-like domain in the extracellular region of RPTP gamma defines a new subfamily of receptor tyrosine phosphatases.” Mol Cell Biol 13(3):1497-506.
Barrett, A. W., A. T. Cruchley, et al. (1996). “Oral mucosal Langerhans' cells.” Crit. Rev Oral Biol Med 7(1): 36-58.
Baumann, I., W. Kolowos, et al. (2002). “Impaired uptake of apoptotic cells into tingible body macrophages in germinal centers of patients with systemic lupus erythematosus.” Arthritis Rheum 46(1): 191-201.
Bendriss-Vermare, N., C. Barthelemy, et al. (2001). “Human thymus contains IFN—alpha—producing CD11c(−), myeloid CD11c(+), and mature interdigitating dendritic cells.” J Clin Invest 107(7): 835-44.
Bruynzeel-Koomen, C., D. F. van Wichen, et al. (1986). “The presence of IgE molecules on epidermal Langerhans cells in patients with atopic dermatitis.” Arch. Dermatol. Res. 278(3): 199-205.
Caux, C., C. Dezutter-Dambuyant, et al. (1992). “GM-CSF and TNF-alpha cooperate in the generation of dendritic Langerhans cells.” Nature 360(6401): 258-61.
de la Fuente-Garcia, M. A., J. M. Nicolas, et al. (1998). “CD148 is a membrane protein tyrosine phosphatase present in all hematopoietic lineages and is involved in signal transduction on lymphocytes.” Blood 91(8): 2800-9.
de Saint-V is, B., J. Vincent, et al. (1998). “A novel lysosome-associated membrane glycoprotein, DC-LAMP, induced upon DC maturation, is transiently expressed in MHC class II compartment.” Immunity 9(3): 325-36.
Fokkens, W., D. Broekhuis-Fluitsma, et al. (1991). “Langerhans cells in nasal mucosa of patients with grass pollen allergy.” Immunobiology 182(2): 135-42.
Fokkens, W. J., J. G. Vinke, et al. (1998). “Differences in cellular infiltrates in the adenoid of allergic children compared with age- and gender-matched controls.” Clin Exp Allergy 28(2): 187-95.
Hart, D. N. J., K. MacDonald, et al. (2001). Phenotipic characterization of dendritic cells. Dendritic Cells, Biology and Clinical Applications, Second Edition. M. T. Lotze and A. W. Thomson. London, San Diego, Academic Press.
Havenith, C. E., A. J. Breedijk, et al. (1993). “T cell priming in situ by intratracheally instilled antigen-pulsed dendritic cells.” Am J Respir Cell Mol Biol 8(3): 319-24.
Katou, F., H. Ohtani, et al. (2003). “Differential expression of CCL19 by DC-Lamp+mature dendritic cells in human lymph node versus chronically inflamed skin.” J Pathol 199(1): 98-106.
Katz, S. I., K. Tamaki, et al. (1979). “Epidermal Langerhans cells are derived from cells originating in bone marrow.” Nature 282(5736): 324-6.
Krummel, M. F. and M. M. Davis (2002). “Dynamics of the immunological synapse: finding, establishing and solidifying a connection.” Curr Opin Immunol 14(1): 66-74.
Lissoni, P., L. Vigore, et al. (1999). “Circulating dendritic cells in early and advanced cancer patients: diminished percent in the metastatic disease.” J Biol Regul Homeost Agents 13(4): 216-9.
Liu, S., Y. Sugimoto, et al. (2003). “Function analysis of estrogenically regulated protein tyrosine phosphatase gamma (PTPgamma) in human breast cancer cell line MCF-7.” Oncogene.
Lotze, M. T. and A. W. Thomson (2001). Dendritic Cells, Biology and Clinical Applications, Second Edition, Academic Press.
MacBeath, G. and S. L. Schreiber (2000). “Printing proteins as microarrays for high-throughput function determination.” Science 289(5485): 1760-3.
Maldonado-Lopez, R., T. De Smedt, et al. (1999), “CD8alpha+ and CD8 alpha—subclasses of dendritic cells direct the development of distinct T helper cells in vivo.” J Exp Med 189(3): 587-92.
Mueller, C. G., M. C. Rissoan, et al. (1997).—“Polymerase chain reaction selects a novel disintegrin proteinase from CD40-activated germinal center dendritic cells.” J Exp Med 186(5): 655-63.
Muzio, M., F. Re, et al. (1994). “Interleukin-13 induces the production of interleukin-1 receptor antagonist (IL-1ra) and the expression of the mRNA for the intracellular (keratinocyte) form of IL-1ra in human myelomonocytic cells.” Blood 83(7): 1738-43.
Novak, N., J. Haberstok, et al. (1999). “Dendritic cells in allergy.” Allergy 54(8): 792-803.
Pawson, T. and J. D. Scott (1997). “Signaling through scaffold, anchoring, and adaptor proteins.” Science 278(5346): 2075-80.
Peters, J. H., R. Gieseler, et al. (1996). “Dendritic cells: from ontogenetic orphans to myelomonocytic descendants.” Immunol Today 17(6): 273-8.
Pulendran, B., J. L. Smith, et al. (1999). “Distinct dendritic cell subsets differentially regulate the class of immune response in vivo.” Proc Natl Acad Sci USA 96(3): 1036-41.
Res, P., E. Martinez-Caceres, et al. (1996). “CD34+CD38dim cells in the human thymus can differentiate into T, natural killer, and dendritic cells but are distinct from pluripotent stem cells.” Blood 87(12): 5196-206.
Rissoan, M. C., V. Soumelis, et al. (1999). “Reciprocal control of T helper cell and dendritic cell differentiation.” Science 283(5405): 1183-6.
Sallusto, F. and A. Lanzavecchia (1994). “Efficient presentation of soluble antigen by cultured human cells is maintained by granulocyte/macrophage colony-stimulating factor plus interleukin 4 and downregulated by tumor necrosis factor alpha.” J Exp Med 179(4): 1109-18.
Sminia, T., M. M. Wilders, et al. (1983). “Characterization of non-lymphoid cells in Peyer's patches of the rat.” Immunobiology 164(2): 136-43.
Soler, P., A. Tazi, et al. (1991). “Pulmonary dendritic cells.” Rev Mal Respir 8(2): 191-6.
Sorio, C., P. Melotti, et al. (1997). “Receptor protein tyrosine phosphatase gamma, Ptpgamma, regulates hematopoietic differentiation.” Blood 90(1): 49-57.
Sorio, C., J. Mendrola, et al. (1995). “Characterization of the receptor protein tyrosine phosphatase gene product PTPgamma: binding and activation by triphosphorylated nucleosides.” Cancer Res 55(21): 4855-64.
Sozzani, S., W. Luini, et al. (1997). “Receptor expression and responsiveness of human dendritic cells to a defined set of CC and CXC chemokines.” J Immunol 159(4): 1993-2000.
Steinman, R. M. and K. Inaba (1999). “Myeloid dendritic cells.” J Leukoc Biol 66(2): 205-8.
Steinman, R. M., D. S. Lustig, et al. (1974). “Identification of a novel cell type in peripheral lymphoid organs of mice. 3. Functional properties in vivo.” J Exp Med 139(6): 1431-45.
Taniguchi, Y., R. London, et al. (1999). “The receptor protein tyrosine phosphatase, PTP-RO, is upregulated during megakaryocyte differentiation and Is associated with the c-Kit receptor.” Blood 94(2): 539-49.
Trautmann, A. and S. Valitutti (2003). “The diversity of immunological synapses.” Curr Opin Immunol 15(3): 249-54.
Van Huijsduijnen, R. H. (1998). “Protein tyrosine phosphatase: counting the trees in the forest.” Gene 255: 1-8.
van Niekerk, C. C. and L. G. Poels (1999). “Reduced expression of protein tyrosine phosphatase gamma in lung and ovarian tumors.” Cancer Lett 137(1): 61-73.
Vandenabeele, S., H. Hochrein, et al. (2001). “Human thymus contains 2 distinct dendritic cell populations.” Blood 97(6): 1733-41.
Vermi, W., R. Bonecchi, et al. (2003). “Recruitment of immature plasmacytoid dendritic cells (plasmacytoid monocytes) and myeloid dendritic cells in primary cutaneous melanomas.” J Pathol 200(2): 255-68.
Yasuda, K., K. Okano, et al. (2000). “Focal extraction of surface-bound DNA from a microchip using photo-thermal denaturation.” Biotechniques 28(5): 1006-11.
Young, I. D., L. Ailles, et al. (1991). “Tanscription of cRNA for in situ hybridization from polymerase chain reaction-amplified DNA.” Lab Invest 64(5): 709-12.

1. A method for identifying myeloid or plasmacytoid dendritic cells provided by a mammal, stimulated or unstimulated, comprising:
a) preparing a cell sample;
b) contacting said cell sample with at least a compound capable of selectively binding a phosphatase of said myeloid or said plasmacytoid dendritic cells to form a complex; and
c) detecting said complex;
wherein said phosphatase is the Receptor type Tyrosine Phosphatase Gamma Protein (“PTPRG”), acting as a specific marker of said dendritic cells; and
wherein said compound is a polypeptide capable of selectively bind to the PTPRG, to a fragment thereof or to a oligonucleotide complementary to a PTPRG mRNA oligonucleotide as to selectively recognize said dendritic cells in said cell sample.

2: Method of identification as claimed in claim 1, wherein said polypeptide is an antibody or a fragment thereof, developed against a polypeptide antigen containing a total or partial amino acid sequence of the human PTPRG sequence present in Genbank database, accession number NM_{—}002841.

3: Method of identification as claimed in claim 2, wherein said antibody, or fragment thereof, is developed against a polypeptide antigen containing an amino acid sequence selected from the group consisting of the sequences:
CAGPTWQDSKLRRWNFHWAHSNGSAGSEHSINGRRF,
CNNFRPQQRL,
CRNQTEPSPTPSSPNRT,
CGSDPKRPEMPSKKPMSRGDRFSED,
GSDPKRPEMPSKKPMSRGDRFSEDC,
GSDPKRPEMPSKKPMSRGDRFSED,
EYLRNNFRPQQRLHDR,
NEDEKEKTFTKDSDKDLK,
CEEGEKDEKSESEDGEREH,
EEDGEKDSEKKEKSGVTH,
SDPKPEMPSKKPMSRGDR,
CPSSGERGEKGSRK,
CKCDQYWP,
CQKGNPKGRQN,
TVDKNGAVLIADESD,
and
SEDGEREHEEDGEKD,

4: The method of identification as claimed in claim 2, wherein step b) is accomplished using a composition comprising an effective amount of at least one of said antibody or fragment thereof with at least one suitable adjuvants, excipients, and solvents.

5: The method of identification as claimed in claim 1, wherein said step of detecting of the antigen—antibody complex is performed applying a technique selected from the group consisting of: cytofluorometry, immunofluorescence on at least one of cells and biological tissues, immunocyte-histochemistry on at least one of cells and biological tissues, ELISA essay, and immunoprecipitation.

6: The method of identification as claimed in claim 1, wherein said oligonucleotides comprise at least a sequence of nucleotides complementary to at least one part of the sequence of mRNA PTPRG nucleotides present in Genbank database, accession number NM_{—}002841, and said step b) comprises hybridization of the nucleic acid of said cells with said complementary nucleotide sequences.

7: The method of identification as claimed in claim 1, wherein said oligonucleotides comprise a plurality of nucleotide sequences complementary to nucleic acid sequences present in databases of myeloid or plasmacytoid dendritic cells expression and at least on mRNA PTPRG nucleotide sequence.

8: The method of identification as claimed in claim 1, wherein step c) further comprises a comparison of a nucleic acid hybridization model of the cells to be identified with a control expression model in comparison databases; and
wherein the expression level of said nucleic acid hybridization model that is substantially similar to the control expression model present in the comparison databases indicates that the sample cells are myeloid or plasmacytoid dendritic cells.

9: The method of identification as claimed in claim 6, wherein said expression level is determined by a technique selected from the group consisting of: northern blot, RT [symbol] PCR, and solid-phase chip.

10: The method of identification as claimed in claim 1, wherein said compound comprises a plurality of antibodies developed against and specifically binding to peptidic compounds selected from the group consisting of: peptides and polypeptides or fragment thereof derived from the PTPRG peptidic sequence.

11: The method of identification as claimed in claim 10, wherein said step of detecting the complex comprises the comparison of an expression model obtained with a control expression model present in comparison databases
and wherein the expression level of said expression model which that is substantially similar to the control expression model in comparison databases indicates that the sample cells are myeloid or plasmacytoid dendritic cells.

12: A method for separating myeloid or plasmacytoid dendritic cells from a cell sample comprising a step of identifying dendritic cells as claimed in claim 11 and positive or negative separating cells that express PTPRG phosphatase.

13: The method of separation as claimed in claim 12, wherein said separating step is carried out applying a technique of magnetic separation or cell sorting with cytofluorimeters, immunoprecipitation, or affinity chromatography.

14: A method comprising measuring the expression level of dendritic cells,
wherein said expression level is determined by detecting a level of the antigen-antibody complex or a level of the hybridization model of nucleic acids of the dendritic cells.

15: The method for in accordance with claim 14, further comprising comparing the expression level
detected with level expressed by comparison and control cells databases,
wherein expression level that is higher in the dendritic cells compared to the expression level of comparison and control cells indicates an activity of dendritic cells higher than the comparison and control cells.

16: A method for identifying an agent capable of modulating an immune response comprising:
determining the PTPRG expression of first nucleic acids without said agent;
exposing one of insulated cells and an organism with said agent;
determining the expression of second nucleic acids specific for PTPRG from the one of insulated cells and an organism;
comparing the expression of the first nucleic acids and the second nucleic acids to verify if said agent is an immunomodulatory agent.

17: The method in accordance with claim 15, further comprising
administering to said dendritic cell an effective amount of at least an immunomodulatory agent having a receptor on the surface of said cells to modulate the activity of said cells;
determining a second activity of said dendritic cells applying the method in accordance with claim 15;
comparing the increase or the decrease of the activity to verify the immunostimulatory or immunoinhibitory action of said agent;
wherein determination of a first activity of said dendritic occurs without use of an immunomodulatory agent.

18: The method as claimed in claim 17, wherein said immunomodulatory agent is selected from the group including antibodies, specific antibody fragments for a receptor, ligands for the receptor, polypeptides containing the sequence of PTPRG gene in a deduced extracellular domain.

19: The method as claimed in claim 17, further comprising inducing an immune response after said administering step by an immunomodulatory agent selected from the group consisting of bacterial agents; viral derivative agents; modified or attenuated pathogens; and cancer cells or derivatives.

20: A method or essay for screening suitable for selecting an immunomodulatory agent or for comparing the immunomodulatory agents ability comprising:
detecting the dendritic cell activity,
modulating the dendritic cell activity, or
identifying an agent able to modulate an immune response in a separate, combined, or sequential manner;
wherein the method is suitable for selecting an immunomodulatory agent or for comparing the immunomodulatory agents ability.

21: The method as claimed in claim 16, wherein said immunomodulatory agent is a pharmaceutically acceptable agent.

22: An agent comprising: an antibody, or fragment thereof, wherein said antibody is developed against an antigen having a polypeptide containing a total or partial amino acid sequence of the human PTPRG sequence present in Genbank database, accession number NM_{—}002841.

23: The agent as claimed in claim 22, developed against an antigen having a polypeptide selected from the group comprising the sequences:
CAGPTWQDSKLRRWNFHWAHSNGSAGSEHSINGRRF,
CNNFRPQQRL,
CRNQTEPSPTPSSPNRL,
CGSDPKRPEMPSKKPMSRGDRFSED,
GSDPKRPEMPSKKPMSRGDRFSED,
GSDPKRPEMPSKKPMSRGDRFSEDC,
CPSSGERGEKGSRK,
CKCDQYWP,
CQKGNPKGRQN,
TVDKNGAVLIADESD,
and
SEDGEREHEEDGEKD
to allow the selective recognizing of said dendritic cells.

24: Antibody, or fragment thereof, as claimed in claim 23, wherein said antibody is monoclonal or polyclonal.

25: The agent as claimed in claim 22, wherein said antibody, or fragment thereof, is chimeric.

26: The agent of claim 22 Composition for carrying out the method as claimed in claims 4, 10 or 20 further comprising an effective amount of one or more antibodies or fragments thereof as claimed in claims 22 to 25 together with at least one suitable adjuvant, and/or excipient, and/or solvent.

27: A solid-phase chip for identifying dendritic cells for carrying out the method in accordance with any of claims from 6 to 9 comprising:
a solid flat substrate; and
a plurality of oligonucleotides, each oligonucleotide having a known sequence and bonded to the substrate at a known position;
wherein said oligonucleotides are complementary to a part of a sequence of nucleic acids of myeloid or plasmacytoid dendritic cells.

28: A solid-phase chip suitable for providing the method in accordance with claim 10, comprising:
a solid flat substrate; and
a plurality of polypeptides bonded to said substrate and located at a known position;
wherein said polypeptides comprise a plurality of antibodies developed against polypeptides selected from the group comprising peptides, polypeptides, or fragment thereof derived from the PTPRG peptidic sequence.

29: A kit for performing the method and/or the screening essay as claimed in claim 20, comprising at least one of:
(a) an agent comprising an antibody, or fragment thereof, wherein said antibody is developed against an antigen having a polypeptide containing a total or
partial amino acid sequence of the human PTPRG sequence present in Genbank database, accession number NM 002841;
(b) a solid-phase chip for identifying dendritic further comprising:
a solid flat substrate, and
a plurality of oligonucleotides, each oligonucleotide having a known sequence and bonded to the substrate at a known position,
wherein said oligonucleotides are complementary to a part of a sequence of nucleic acids of myeloid or plasmacytoid dendritic cells; and
(c) a solid-phase chip further comprising:
a solid flat substrate; and
a plurality of polypeptides bonded to said substrate and located at a known position;
wherein said polypeptides comprise a plurality of antibodies developed against polypeptides selected from the group comprising peptides, polypeptides, or fragment thereof derived from the PTPRG peptidic sequence;
together with at least one suitable adjuvant, excipient, solvent, stainer.

30: A pharmaceutical composition comprising an effective amount of an immunomodulatory agent identified in accordance with the method claimed in claim 16 with suitable pharmaceutically acceptable excipients.

31: A pharmaceutical composition as claimed in claim 30, wherein said immunomodulatory agent comprises modified antisense oligonucleotides complementary to and hybridizable with nucleic acids corresponding to the PTPRG marker with suitable pharmaceutically acceptable excipients.

32: Use of an effective amount of a pharmaceutical composition as claimed in claim 30, for preparing a drug for modulating the immune response to an infection or to a autoimmune disorder.

33. Use of an effective amount of a pharmaceutical composition as claimed in 31, for preparing a drug for modulating the immune response to an infection or to a autoimmune disorder.

34. The method of identification as claimed in claim 3, wherein step b) is accomplished using a composition comprising an effective amount of at least one of said-antibody or fragment thereof with at least one suitable adjuvants, excipients, and solvents.

35. The method of identification as claimed in claim 7, wherein said expression level is determined by a technique selected from the group consisting of: northern blot, RT [symbol] PCR, solid-phase chip.

36: The method of identification as claimed in claim 8, wherein said expression level is determined by a technique selected from the group consisting of: northern blot, RT [symbol] PCR, solid-phase chip.

37: The method as claimed in 17, wherein said immunomodulatory agent is a pharmaceutically acceptable agent.

38: The method as claimed in claim 18, wherein said immunomodulatory agent is a pharmaceutically acceptable agent.

39: The method as claimed in claim 19, wherein said immunomodulatory agent is a pharmaceutically acceptable agent.

40: The method as claimed in claim 20, wherein said immunomodulatory agent is a pharmaceutically acceptable agent.

41: The agent as claimed in claim 23, wherein said antibody is chimeric.

42: The agent of claim 23 further comprising an effective amount together with at least one suitable adjuvant, excipient, or solvent.

43: The agent of claim 24 further comprising an effective amount together with at least one suitable adjuvant, excipient, or solvent.

44: The agent of claim 25 further comprising an effective amount together with at least one suitable adjuvant, excipient, or solvent.

45: The solid-phase chip for identifying dendritic cells of claim 27, wherein at least on nucleic acid comprises mRNA PTPRG nucleotides according to the sequence present in Genbank database, accession number NM_{—}002841.

46: The solid-phase chip of claim 28, wherein the PTPRG peptidic sequence is SEQ ID NO:2.

47: The method of identification as claimed in claim 2, wherein said step of detecting of the antigen—antibody complex is performed applying a technique selected from the group consisting of: cytofluorometry, immunofluorescence on at least one of cells and biological tissues, immunocyte-histochemistry on at least one of cells and biological tissues, ELISA essay, and immunoprecipitation.

48: The method of identification as claimed in claim 3, wherein said step of detecting of the antigen—antibody complex is performed applying a technique selected from the group consisting of: cytofluorometry, immunofluorescence on at least one of cells and biological tissues, immunocyte-histochemistry on at least one of cells and biological tissues, ELISA essay, and immunoprecipitation.

49: The method of identification as claimed in claim 4, wherein said step of detecting of the antigen—antibody complex is performed applying a technique selected from the group consisting of: cytofluorometry, immunofluorescence on at least one of cells and biological tissues, immunocyte-histochemistry on at least one of cells and biological tissues, ELISA essay, and immunoprecipitation.


SEQUENCE LISTING (18 sequences)

SEQ ID NO: 1 - DNA, 5787 bp, Homo sapiens
atgttacttcctgtatggaggcatggccagtttccagcgccgcgctcttcgttccttcccagcctgcgccggagccacaactttcaggagcatggactgaaggcgccctcgccccagcgcccctctgagatcctttgtgttttcctccgtttcctccggccgtttctattttggggggctctccgctccccctgcctctcccctccccttcccctctcgcaaacatgcctccttccttcccggggccctggaaggagctgcctgcctgaagcccggagacgccgcgccgcgctcagccccgccgccgcccgccggctctcgggctgtgctgcgctgccgactcaagttggggatcctcggctgctcgccgccgccgcccgcggtccctgtctgccccaggcccggggcatcgccgccggccgccgactccgcgccctgcccgatcggctctctcctttttaaacggaaagcagcctttctccgccgagaggatcgtccccagcgtggctctgcgttcccggtcactttttgagattttccggggggcgctcggcggcttcccggattccaaggggactcgggccgccgagcgcggggggcccgtggagcgggcgagccggggaagcgccccggcttagcggaggctcgcacggaggcaagaacttattcaacaagtttacctccctgctttcctcttttcgatgtgcgttttcggacatgcggaggttactggaaccgtgttggtggattttgttcctgaaaatcaccagttccgtgctccattatgtcgtgtgcttccccgcgttgacagaaggctacgttggggccctgcacgagaatagacacggcagcgcagtgcagatccgcaggcgcaaggcttcaggcgacccgtactgggcctactctggtgcctatggtcctgagcactgggtcacgtctagtgtcagctgtgggagccgtcaccagtctcctattgacattttagaccagtatgcgcgtgttggggaagaataccaggaactgcaactcgatggcttcgacaatgagtcttctaacaaaacctggatgaaaaacacagggaaaacagtcgccatccttctgaaagacgactattttgtcagtggagctggtctacctggcagattcaaagctgagaaggtggaatttcactggggccacagcaatggctcagcgggctctgaacacagcatcaatggcaggaggtttcctgttgagatgcagattttcttttacaatccagatgactttgacagctttcaaaccgcaatttctgagaacagaataatcggagccatggccatattttttcaagtcagtccgagggacaattctgcactggatcctattatccacgggttgaagggtgtcgtacatcatgagaaggagacctttctggatcctttcgtcctccgggacctcctgcctgcatccctgggcagctattatcggtacacaggttccttgaccacaccaccgtgtagcgaaatagtggagtggatagtcttccggagacccgtccccatctcttaccatcagcttgaggctttttattccatcttcaccacggagcagcaagaccatgtcaagtcggtggagtatctgagaaataactttcgaccacagcagcgtctgcatgacagggtggtgtccaagtccgccgtccgtgactcctggaaccacgacatgacagacttcttagaaaacccactggggacagaagcctctaaagtttgcagctctccacccatccacatgaaggtgcagcctctgaaccagacggcactgcaggtgtcctggagccagccggagactatctaccacccacccatcatgaactacatgatctcctacagctggaccaagaatgaggacgagaaggagaagacgtttacaaaggacagcgacaaagacttgaaagccaccattagccatgtctcacccgatagcctttacctgttccgagtccaggccgtgtgtcggaacgacatgcgcagcgactttagccagacgatgctgtttcaagctaataccactcgaatattccaagggaccagaatagtgaaaacaggagtgcccacagcgtctcctgcctcttcagccgacatggcccccatcagctcggggtcttctacctggacgtcctctggcatcccattctcatttgtttccatggcaactgggatgggcccctcctccagtggcagccaggccacagtggcctcggtggtcaccagcacgctgctcgccggcctggggttcggcggtggtggcatctcctctttccccagcactgtgtggcccacgcgcctcccgacggccgcctcagccagcaagcaggcggctaggccagtcctagccaccacagaggccttggcttctccagggcccgatggtgattcgtcaccaaccaaggacggcgagggcaccgaggaaggagagaaggatgagaaaagcgagagtgaggatggggagcgggagcacgaggaggatggagagaaggactccgaaaagaaggagaagagtggggtgacccacgctgccgaggagcggaatcagacggagcccagccccacaccctcgtctcctaacaggactgccgagggagggcatcagactatacctgggcatgagcaggatcacactgccgtccccacagaccagacgggcggaaggagggatgccggcccaggcctggaccccgacatggtcacctccacccaagtgccccccaccgccacagaggagcagtatgcagggagtgatcccaagaggcccgaaatgccatctaaaaagcctatgtcccgcggggaccgattttctgaagacagcagatttatcactgttaatccagcggaaaaaaacacctctggaatgataagccgccctgctccagggaggatggagtggatcatccctctgattgtggtatcagccttgaccttcgtgtgcctcatccttctcattgctgtgctcgtttactggagagggtgtaacaaaataaagtccaagggctttcccagacgtttccgtgaagtgccttcttctggggagagaggagagaaggggagcagaaaatgttttcagactgctcatttctatgtggaagacagcagttcacctcgagtggtccctaatgaaagtattcctattattcctattccggatgacatggaagccattcctgtcaaacagtttgtcaaacacatcggtgagctctattctaataaccagcatgggttctctgaggattttgaggaagtccagcgctgtactgctgatatgaacatcactgcagagcattccaatcatccagaaaacaagcacaaaaacagatacatcaacattttagcatatgatcacagtagggtgaagttaagacctttaccaggaaaagactctaagcacagcgactacattaatgcaaactatgttgatggttacaacaaagcaaaagcctacattgccacccaaggacctttgaagtctacatttgaagatttctggaggatgatttgggaacaaaacactggaatcattgtgatgattacgaaccttgtggaaaaaggaagacgaaaatgtgatcagtattggccaacagagaacagtgaggaatatggaaacattattgtcacgctgaagagcacaaaaatacatgcctgctacactgttcgtcgtttttcaatcagaaatacaaaagtgaaaaagggtcagaagggaaatcccaagggtcgtcagaatgaaagggtagtgatccagtatcactatacacagtggcctgacatgggagttcccgagtatgcccttccagtactgactttcgtgaggagatcctcagcagctcggatgccagaaacgggccctgtgttggtgcactgcagtgctggtgtgggcagaacaggcacctatattgtaatagacagcatgctgcaacagataaaagacaaaagcacagttaacgtcctgggattcctgaagcatatcaggacacagcgtaactacctcgtccagactgaggagcagtacattttcatccatgatgccttgttggaagccattcttggaaaggagactgaagtatcttcaaatcagctgcacagctatgttaacagcatccttataccaggagtaggaggaaagacacgactggaaaagcaattcaagctggtcacacagtgtaatgcaaaatatgtggaatgtttcagtgctcagaaagagtgtaacaaagaaaagaacagaaactcttcagttgtgccatctgagcgtgctcgagtgggtcttgcaccattgcctggaatgaaaggaacagattacattaatgcttcttatatcatgggctattataggagcaatgaatttattataactcagcatcctctgccacatactacgaaagatttctggcgaatgatttgggatcataacgcacagatcattgtcatgctgccagacaaccagagcttggcagaagatgagtttgtgtactggccaagtcgagaagaatccatgaactgtgaggcctttaccgtcacccttatcagcaaagacagactgtgcctctctaatgaagaacaaattatcatccatgactttatccttgaagctacacaggatgactatgtcttagaagttcggcactttcagtgtcccaaatggcctaacccagatgcccccataagtagtacctttgaacttatcaacgtcatcaaggaagaggccttaacaagggatggtcccaccattgttcatgatgagtatggagcagtttcagcaggaatgttatgtgcccttaccaccctgtcccagcaactggagaatgaaaatgctgtggatgttttccaggttgcaaaaatgatcaatcttatgaggcctggagtattcacagacattgaacaataccagttcatctataaagcaaggcttagcttggtcagcactaaagaaaatggaaatggtcccatgacagtagacaaaaatggtgctgttcttattgcagatgaatcagaccctgctgagagcatggagtccctagtgtgactggaatcctgaaagggcacttaatttgtaaacttctgaagactgagaacttttttgaggccttttttgccagactctaggttatacaataacccagttacttttttacactgataaaagttttgatatttattttttgccattttatgtcttaatggtatcctactgagcatttgcacctctgttcatttcacacagtgaaacgcaattttacctagtttgcactatatgatcagtgttactgcctataatcttatacaacagcaaaccctgatgtgacattccatgacgacatacatgctacttttttttagttcaatacagtgaaggtctttgttatgacagtgaatattgcttttattattattattgctgaagtggttgcattctactagcaggcaatgctgtacttttcttcagtcctcctctcctttttattttaggcactgttcaatactgtatgccttctgtattttaatggagtggatagcattgttttcttttacagactagcaggctactgggacctaaaaaggtctgttaatgtcatggccttgaaacagttccatttatgctggttaagagatcccttaagaagttagaaggcttaagaactgcttcatgtgaacatcccttattagttacaaagttatattcacagttttttaaaaatgtgtcaaaataaaggataactctgtattacagctttcccagtaaaaaaaaaaaaaaaaa

SEQ ID NO: 2 - protein, 1445 aa, Homo sapiens
MRRLLEPCWWILFLKITSSVLHYVVCFPALTEGYVGALHENRHGSAVQIRRRKASGDPYWAYSGAYGPEHWVTSSVSCGSRHQSPIDILDQYARVGEEYQELQLDGFDNESSNKTWMKNTGKTVAILLKDDYFVSGAGLPGRFKAEKVEFHWGHSNGSAGSEHSINGRRFPVEMQIFFYNPDDFDSFQTAISENRIIGAMAIFFQVSPRDNSALDPIIHGLKGVVHHEKETFLDPFVLRDLLPASLGSYYRYTGSLTTPPCSEIVEWIVFRRPVPISYHQLEAFYSIFTTEQQDHVKSVEYLRNNFRPQQRLHDRVVSKSAVRDSWNHDMTDFLENPLGTEASKVCSSPPIHMKVQPLNQTALQVSWSQPETIYHPPIMNYMISYSWTKNEDEKEKTFTKDSDKDLKATISHVSPDSLYLFRVQAVCRNDMRSDFSQTMLFQANTTRIFQGTRIVKTGVPTASPASSADMAPISSGSSTWTSSGIPFSFVSMATGMGPSSSGSQATVASVVTSTLLAGLGFGGGGISSFPSTVWPTRLPTAASASKQAARPVLATTEALASPGPDGDSSPTKDGEGTEEGEKDEKSESEDGEREHEEDGEKDSEKKEKSGVTHAAEERNQTEPSPTPSSPNRTAEGGHQTIPGHEQDHTAVPTDQTGGRRDAGPGLDPDMVTSTQVPPTATEEQYAGSDPKRPEMPSKKPMSRGDRFSEDSRFITVNPAEKNTSGMISRPAPGRMEWIIPLIVVSALTFVCLILLIAVLVYWRGCNKIKSKGFPRRFREVPSSGERGEKGSRKCFQTAHFYVEDSSSPRVVPNESIPIIPIPDDMEAIPVKQFVKHIGELYSNNQHGFSEDFEEVQRCTADMNITAEHSNHPENKHKNRYINILAYDHSRVKLRPLPGKDSKHSDYINANYVDGYNKAKAYIATQGPLKSTFEDFWRMIWEQNTGIIVMITNLVEKGRRKCDQYWPTENSEEYGNIIVTLKSTKIHACYTVRRFSIRNTKVKKGQKGNPKGRQNERVVIQYHYTQWPDMGVPEYALPVLTFVRRSSAARMPETGPVLVHCSAGVGRTGTYIVIDSMLQQIKDKSTVNVLGFLKHIRTQRNYLVQTEEQYIFIHDALLEAILGKETEVSSNQLHSYVNSILIPGVGGKTRLEKQFKLVTQCNAKYVECFSAQKECNKEKNRNSSVVPSERARVGLAPLPGMKGTDYINASYIMGYYRSNEFIITQHPLPHTTKDFWRMIWDHNAQIIVMLPDNQSLAEDEFVYWPSREESMNCEAFTVTLISKDRLCLSNEEQIIIHDFILEATQDDYVLEVRHFQCPKWPNPDAPISSTFELINVIKEEALTRDGPTIVHDEYGAVSAGMLCALTTLSQQLENENAVDVFQVAKMINLMRPGVFTDIEQYQFIYKARLSLVSTKENGNGPMTVDKNGAVLIADESDPAESMESLV

SEQ ID NO: 3 - protein, 36 aa, Homo sapiens
CAGPTWQDSKLRRWNFHWAHSNGSAGSEHSINGRRF

SEQ ID NO: 4 - protein, 10 aa, Homo sapiens
CNNFRPQQRL

SEQ ID NO: 5 - protein, 17 aa, Homo sapiens
CRNQTEPSPTPSSPNRT

SEQ ID NO: 6 - protein, 24 aa, Homo sapiens
CGSDPKRPEMPSKKPMSRGDRFED

SEQ ID NO: 7 - protein, 25 aa, Homo sapiens
GSDPKRPEMPSKKPMSRGDRFSEDC

SEQ ID NO: 8 - protein, 16 aa, Homo sapiens
EYLRNNFRPQQRLHDR

SEQ ID NO: 9 - protein, 18 aa, Homo sapiens
NEDEKEKTFTKDSKDDLK

SEQ ID NO: 10 - protein, 19 aa, Homo sapiens
CEEGEKDEKSESEDGEREH

SEQ ID NO: 11 - protein, 18 aa, Homo sapiens
EEDGEKDSEKKEKSGVTH

SEQ ID NO: 12 - protein, 18 aa, Homo sapiens
SDPKPEMPSKKPMSRGDR

SEQ ID NO: 13 - protein, 14 aa, Homo sapiens
CPSSGERGEKGSRK

SEQ ID NO: 14 - protein, 8 aa, Homo sapiens
CKCDQYWP

SEQ ID NO: 15 - protein, 11 aa, Homo sapiens
CQKGNPKGRQN

SEQ ID NO: 16 - protein, 15 aa, Homo sapiens
TVDKNGAVLIADESD

SEQ ID NO: 17 - protein, 15 aa, Homo sapiens
SEDGEREHEEDGEKD

SEQ ID NO: 18 - protein, 24 aa, Homo sapiens
GSDPKRPEMPSKKPMSRGDRFSED